(19) United States
(12) Patent Application Publication
(10) Pub. No.: US 2026/0118358 A1
(43) Pub. Date: Apr. 30, 2026

(54) MEANS AND METHODS FOR ANTIBODY DISCOVERY

(71) Applicant: LUMICKS CA Holdings B.V., Amsterdam (NL)

(72) Inventors: Andrea CANDELLI, Amsterdam (NL); Rogier Martijn REIJMERS, Amsterdam (NL)

(21) Appl. No.: 19/115,474

(22) PCT Filed: Oct. 16, 2023
(86) PCT No.: PCT/EP2023/078682

§ 371 Date: Mar. 26, 2025

(30) Foreign Application Priority Data
Oct. 17, 2022 (EP) 22201965.5
Oct. 17, 2022 (EP) 22201969.7

(51) Int. Cl. G01N 33/569; C12N 5/0781

(52) U.S. Cl. CPC G01N 33/56972; C12N 5/0635

(57) ABSTRACT

The current invention relates to cell-cell interaction and in particular to cellular avidity. Provided are improved means and methods to study cell-cell interaction and characterizing cellular avidity. Highly advantageously, it was shown that cellular avidity methods allowed to enrich for B cells expressing an antigen-specific antibody from B cells obtained from subjects exposed to said antigen, e.g. via infection, vaccination, or immunization. Hence, improved means and methods for use in antibody discovery and development are provided. Highly advantageously, it was also shown that cellular avidity methods allowed to determine immune responses against antigens from B cells obtained from subjects exposed to said antigen, e.g. via infection, vaccination, or immunization. Hence, improved means and methods for determining immune response are also provided.

### INTRODUCTION

Antibodies are a critical component of the adaptive immune response for the neutralization and destruction of disease-causing molecules, viruses and cells. Upon exposure to a foreign antigen, the immune system first mounts a diverse polyclonal B cell response, producing a repertoire of antibodies that can recognize multiple antigenic epitopes. Initial antibodies have low binding affinities, activated antibody-producing B cells subsequently undergo a conserved affinity maturation process through somatic hypermutation and clonal selection to generate antibodies with improved affinities against specific target antigens. Antibodies have been further enhanced and engineered to provide for highly advantageous antibody biotherapeutics. Antibody therapeutics have provided significant advancements, in particular in the field of cancer, auto-immune diseases and infectious disease. Hundreds of antibodies have been tested in clinical trials, with about 80 being approved for medical use. For many indications, antibodies have become the dominant treatment modality.

Fundamental to antibody-therapeutics is antibody discovery. The antibody discovery field has relied on methodologies utilizing hybridoma, phage display, transgenic animals carrying human variable regions, up to using B cells obtained from humans. In particular due to the emergence of high throughput technologies, for example based on single B cells, as well as (single) cell sequencing technologies, B cells, e.g. obtained from patients or the like, have proven to be a valuable source for discovering paired heavy and light chain antibody sequences of B cells of interest, useful for example in immunotherapy. Despite the technological advancements, means and methods in the art for antibody discovery still remain cumbersome.

In the light the above, there is still a need in the art to provide for means and methods that can further improve antibody discovery. The current invention now provides for further improved means and methods that are highly useful for the antibody field. In particular, the current invention provides for highly advantageous means and methods for the selection of B cells that express antigen-specific B cell receptors and which can highly efficiently engage with antigens presented by target cells.

In addition, important for assessment of e.g. vaccinations and infection status, is to provide for means and methods that provide for meaningful information regarding immune response and the presence or absence of antibodies. Immune responses can be monitored by assessing immune response, e.g. by assessing the presence of antibodies in blood serum. Infection status can be monitored e.g. by detection of the presence of foreign genetic material (i.a. detected by PCR), and by monitoring the presence of specific foreign proteins (e.g. with an antibody), and by determining the presence of antibodies against a specific foreign protein (e.g. with a serological test).

In the light of the above, there is thus still a need in the art for further means and methods for assessing immune responses, e.g. when a subject is suspected of having an infection, currently or have had in the past. The current invention now provides for further means and methods that are highly useful for determining an immune response. In particular, the current invention provides for highly advantageous means and methods for detecting B cells that express antigen-specific B cell receptors and which can highly efficiently engage with antigens presented by target cells.

### SUMMARY OF THE INVENTION

The current invention shows that highly advantageously, the cellular avidity between a B cell and a target cell presenting an antigen can be used to enrich for B cells that express a B cell receptor (BCR) against an antigen. This way, B cells having an effective interaction with a target antigen presented by a target cell in a similar fashion as occurring in nature can be selected by exerting a force on the B cells away from the target cells, such that B cells that bind less effective to the target cell can be removed.

Hence, the current invention now provides for a highly advantageous method for manipulating B cells that express B cell receptors. By utilizing cellular avidity and applying a force, B cells can be highly effectively selected that express B cell receptors, and subsequently provide for antibodies derived therefrom, that have a good interaction with antigens presented by a target cell. This way, advantageously, by presenting an antigen in its naturally occurring constellation, antibodies with good target avidity/affinity can be ultimately obtained, and one does not need to e.g. resort to cumbersome engineering of antigens to ensure proper presentation of epitopes, when presenting soluble antigens or antigens attached to e.g. a bead, which in particular is different, and can be problematic, for membrane proteins and the like, which have strong hydrophobic regions. Furthermore, by subsequently providing the B cells that have remained attached with e.g. a labelled antigen, B cells that have had an effective interaction with the target cells and expressing a BCR against the target antigen can be identified. This is because B cells that aspecifically bind to a target cell or that bind to another antigen presented by the target cells would not be provided with the label.

Hence, in one embodiment, the current invention provides for a method for manipulating B cells expressing antigen-specific BCRs comprising the steps of:
a) providing B cells obtained from a subject exposed to an antigen,
b) providing target cells expressing the antigen attached to a surface,
c) contacting the B cells with the target cells attached to a surface to allow the B cells to interact with the target cells, and
d) exerting a force on the B cells, wherein the force is in a direction away from the target cells.

The method steps as described above are highly useful in means and methods for antibody discovery. For example, these method steps are highly useful for enriching for B cells expressing antigen-specific BCRs, e.g. when subsequently collecting B cells that remain bound to the target cells. These method steps are also highly useful for identifying B cells expressing antigen-specific BCRs, e.g. by subsequently providing the B cells that remain bound to the target cells with a suitable label representative of the antigen presented by the target cells, or by methods employing sequencing of variable regions of B cells that remain bound to the target cells. The enriched B cells and/or sequences of the variable regions of the BCRs may be used for subsequently producing antibodies of interest. Non-limiting examples include preparing hybridoma's of enriched B cells and/or use the sequences, in particular the complementarity determining region (CDR) sequences, to generate expression constructs for production of antibodies in cells.

The means and methods as outlined herein are useful for the manipulation of B cells and are highly useful in the field of diagnostics. As shown in the example section, the means and methods in accordance with the invention allowed to identify and/or confirm patients previously exposed to the antigen. The current application now also provides for a highly advantageous method for manipulating B cells that express B cell receptors. By utilizing cellular avidity and applying a force, B cells that remain bound can be determined that provide a good interaction with antigens presented by a target cell. This way, advantageously, by presenting an antigen in its naturally occurring constellation, an assessment of B cells with proper target avidity can be made. Furthermore, by subsequently providing the B cells that have remained attached with e.g. a labelled antigen, B cells that have had an effective interaction with the target cells and expressing a BCR against the target antigen can be identified, because B cells that aspecifically bind or that bind to another antigen presented by the target cells will not be provided with the label. This way, cellular avidity as determined between B cells and target cells expressing a defined antigen, provides for a quantifiable measure indicative of an effective immune response.

Hence, as such, the steps of the methods as outlined herein can advantageously be applied in diagnostics applications or the like, wherein it is of importance to determine whether or not a subject has mounted a specific immune response against a foreign antigen. For example, patients may be suspected of being infected with a known pathogen. By obtaining B cells from the patient and subjecting the B cells to methods such as outlined in the example section, infection status may be confirmed. Moreover, effectiveness of vaccination strategies e.g. as prophylaxis or in treatments, e.g. against pathogens and/or cancers, may be monitored utilizing the (steps of) the means and methods as outlined herein. In addition, in case of transplant patients, e.g. patients receiving an organ or cell product (derived) from a donor, it may be of interest to monitor whether or not a subject is reacting to the transplant.

In one embodiment, a method of determining an immune response against an antigen is provided comprising the steps of:
a) providing B cells obtained from a subject exposed to an antigen or suspected or being investigated of being exposed to said antigen,
b) providing target cells expressing the antigen attached to a surface,
c) contacting the B cells with the target cells attached to a surface to allow the B cells to interact with the target cells,
d) exerting a force on the B cells, wherein the force is in a direction away from the target cells, and
e) analysing the B cells that remain attached to the target cells.

It is understood that as outlined herein, analysing the B cells that remain attached to the target cells includes preferably the percentage of B cells that remain attached. In order to determine an effective immune response, B cells obtained from the subject may be divided into memory B cells and non-memory B cells and the B cell numbers of each fraction remaining determined in step d) and compared. When the percentage of B cells remaining bound of the memory B cells substantially exceeds the percentage of B cells remaining bound of the non-memory B cells, this can be determined as an indication which confirms infection and/or successful vaccination. Likewise, B cells, preferably memory B cells, can be divided into two fractions, one being incubated with a blocking antigen and the other not, and both fractions being subjected to the method as outlined above. When the percentage of B cells (e.g. memory B cells) that remains bound when not pre-incubated with blocking antigen substantially exceeds the percentage when pre-incubated with blocking antigen, this can be regarded a confirmation of infection and/or successful vaccination. Of course, one can also make in the analysis a comparison with control B cells, which are provided e.g. of a subject known to not have been exposed to the antigen, or one can compare in the analysis the result with control values of further control subjects with known immunological status with regard to the antigen. This way, whether or not an immune response has been triggered can be determined in subjects.

Hence, in a further embodiment, a method of determining an immune response against an antigen is provided comprising the steps of:
a) providing B cells obtained from a subject exposed to an antigen or suspected or being investigated of having been and/or being exposed to said antigen,
b) separating the B cells into memory B cells and non-memory B cells,
c) providing target cells expressing the antigen attached to a surface,
d) contacting the memory B cells with the target cells attached to a surface to allow the memory B cells to interact with the target cells,
e) exerting a force on the memory B cells, wherein the force is in a direction away from the target cells,
f) perform steps d) and e) with the non-memory B cells, and
e) analysing the memory B cells and the non-memory B cells that remain attached to the target cells.

It is understood that such an analysis may involve quantifying the memory B cells and non-memory B cells (e.g. percentage or relative amount remaining) and determining the difference between the amounts of memory B cells and non-memory B cells.

In another embodiment, a method of determining an immune response against an antigen is provided comprising the steps of:
a) providing B cells, preferably memory B cells, obtained from a subject exposed to an antigen or suspected of being exposed to said antigen,
b) providing target cells expressing the antigen attached to a surface,
c) contacting the B cells provided in step a) with the target cells attached to a surface to allow the B cells to interact with the target cells,
d) exerting a force on the B cells, wherein the force is in a direction away from the target cells,
e) repeat steps c) and d) with the B cells provided in step a), wherein the B cells were subjected to a blocking antigen prior to performing steps c) and d) therewith, and
e) analysing the B cells, with and without blocking antigen, that remain attached to the target cells.

It is understood that the analysis may involve quantifying the B cells (e.g. percentage or relative amount remaining) with and without pre-incubation with blocking antigen and determining the difference between each of the amounts determined.

Of course, as said, in further embodiments such as described herein, the analysis may include a comparison with a reference B cell sample instead, e.g. of a subject known and confirmed to have surmounted an effective immune response against the antigen (or of course known not to). The analysis may also include further steps utilizing (labelled) antigens in MACS or FACS analysis or the like. It is understood that these methods may be regarded or do not need to be methods involving enrichment, purification or selection of B cells expressing antigen-specific BCRs, as all that may be required is an analysis of the cells that remain bound after the force has been exerted. Needless to say, it may be contemplated to do include such further steps of enrichment, purification or selection and further analysis, which may be useful in gaining a more in depth understanding of the immune response which is, or may not be, induced. Such more in-depth analysis may also be included for diagnostic purposes.

The means and methods as described herein are highly advantageous for the development of vaccination strategies. Moreover, this is highly advantageous in e.g. the treatment of patients suspected or known of being infected with a pathogen. When e.g. a subject has been diagnosed to be infected with a known pathogen, and the subject is known to have surmounted an effective immune response or not, such information may guide treatment of the subject.

Of course, the means and methods may also be used to determine cross reactivity in case of emerging variants of a particular pathogen. For example, when patients have been vaccinated against a defined pathogen, and of which a further variant emerges, the means and the methods as described herein may subsequently be used to determine whether or not the (memory) B cells are similarly capable of interacting with the further variant antigens and to assess whether or not further complementary vaccination strategies with the new variants may be warranted.

### FIGURES

FIG. 1. Plot of B cell binding to target cells expressing spike protein. A) Memory B cells and non-memory B cells obtained from human subjects of a population highly infected with COVID-19 were incubated with target cells expressing spike protein. The percentage of B cells remaining attached (y-axis) is plotted against the exerted force. The memory B cells (upper line in the plot) shows a higher percentage of binding to the target cells as compared the non-memory B cells. B) In this plot now the memory B cells or non-memory B cells were first incubated with spike protein, before incubating the B cells with the target cells expressing spike protein. The percentage of B cells remaining attached (y-axis) is plotted against the exerted force. The memory B cells (lower line in the plot) show a somewhat lower or similar percentage of binding to the target cells as compared to the non-memory B cells. The memory B cells in the FIG. 1B plot is now much lower as compared to the FIG. 1A plot, likely due to blockage of BCRs capable of binding spike protein expressed by the target cells.

FIG. 2. At the end of a B cell-target cell avidity run (without using pre-incubation/blocking B cells with spike protein), cells remaining were provided with a labelled spike protein and B cells bound to the target cells were detected (indicated with an arrow).

### DETAILED DESCRIPTION

In a first embodiment, a method is provided for manipulating B cells comprising the steps of:
a) providing B cells obtained from a subject exposed to an antigen,
b) providing target cells expressing the antigen attached to a surface,
c) contacting the B cells with the target cells attached to a surface to allow the B cells to interact with the target cells, and
d) exerting a force on the B cells, wherein the force is in a direction away from the target cells.

In another embodiment, a method is provided for manipulating B cells comprising the steps of:
a) providing B cells obtained from a subject,
b) providing target cells expressing the antigen attached to a surface,
c) contacting the B cells with the target cells attached to a surface to allow the B cells to interact with the target cells, and
d) exerting a force on the B cells, wherein the force is in a direction away from the target cells.

As said, the current invention now provides for means and methods that can be utilized to manipulate B cells, in particular of B cells obtained from a subject exposed to an antigen. When a subject, for example a human subject, has been exposed to an antigen, B cells develop in the subject which produce B cell receptors that can recognize the antigen. These B cells have the capacity to further develop into antibody producing cells. The subject will retain B cells, e.g. memory B cells, that express B cell receptors that can recognize the antigen, and which are capable of developing into antibody producing plasma B cells when the subject e.g. becomes again exposed to the antigen.

Subjects can be exposed to an antigen via various means. For example, subjects can be exposed to a vaccine, or be naturally infected, in case of pathogens. A vaccine can comprise a live or dead pathogen. A vaccine preferably comprises a protein, e.g. from the pathogen against which antibodies are to be induced or of an antigen of interest, e.g. a cancer antigen. A vaccine can be an attenuated virus, or a conventional vaccine, i.e. combining an antigen or an inactivated pathogen with a suitable adjuvant. A vaccine can also be a DNA/RNA vaccine or based on a viral vector (e.g. adenovirus-based), capable of expressing the antigen. Pathogens can be algae, bacteria, fungi, prions, viroids, viruses, and parasites. It is understood that immunization or vaccination are terms that can be used interchangeably and mean the same, i.e. administering to a subject a vaccine which results in an immune response against an antigen provided by/with the vaccine. With regard to the subject, it is understood that these include vertebrates, for example mammals, that have a B cell repertoire. For example, as described in the example section, B cells can be obtained from subjects that have been, or are suspected to have been, exposed to antigen, either through natural infection and/or through vaccination.

Nevertheless, in an alternative embodiment, it may also be contemplated to provide for B cells obtained from a subject, which subject is e.g. not known to have been exposed to said antigen which subject may be regarded to be naïve with regard to said antigen. Hence, in such an alternative embodiment, a method is provided for manipulating B cells comprising the steps of:
a) providing B cells obtained from a naïve subject,
b) providing target cells expressing the antigen attached to a surface,
c) contacting the B cells with the target cells attached to a surface to allow the B cells to interact with the target cells, and
d) exerting a force on the B cells, wherein the force is in a direction away from the target cells.

Such an embodiment may be advantageous e.g. when no subjects are available that have been exposed to said antigen, although the number of B cells capable of having an effective interaction with the target antigen may be much reduced (when comparing naïve subjects with exposed subjects). In such an embodiment, one may still enrich for B cells having BCRs capable of interacting with the target antigen, which may be expected to be present in lower numbers and/or interacting less strong and/or less specific with the antigen presented by the target cell.

Target cells expressing the antigen may be any cell presenting the antigen at its cell surface. For example, in case of a receptor which presents at the cell surface, a cell may be transfected or transduced with a vector construct to express said receptor. As described in the example section, a target cell may be e.g. a human cell line which e.g. attaches well to a surface. Of course, primary cells may be also be used. Cells may also be selected that present said antigen naturally. Suitable cells may include eukaryotic cells or prokaryotic cells. Cells that may be selected include fungal cells, animal cells, insect cells, mammalian cells, bacterial cells, yeasts, and protozoa. It may be preferred to select human cells as human targets may be of particular interest in accordance with the invention.

As said, the cells can be attached to a surface. It is understood that surfaces for attaching cells may be any surface suitable for attaching cells. Suitable surfaces for attaching cells include plastic or glass surfaces. These surfaces may be coated e.g. with a protein to promote attachment of cells to the surface, such as poly-L-lysine or the like. Suitable polypeptides for attachment of target cells that may be contemplated and are known in the art include for example fibronectin, poly-L-lysine, poly-D-lysine, poly-L-ornithine, laminin, collagen, fibronectin, fibrinogen, vitronectin, or osteopontin. In any case, a suitable polypeptide or other suitable coating may be selected such that the target cells that are attached to the surface allow for the target cells to remain attached to the surface when applying a force on the B cells. In other words, when a force is applied on these cells which allows for detachment of (some) of these cells from the target cells, the target cells are to substantially remain attached to the surface.

Next, the B cells are contacted with the target cells attached to a surface to allow the B cells to interact with the target cells. It is understood that the B cells, e.g. as obtained from a subject, are heterogeneous with regard to the BCR, i.e. each B cell expresses a defined B cell receptor and between B cells, B cells may express different B cell receptors. Different B cells expressing different BCRs are understood to be comprised in the provided B cells that are capable of interacting with the target antigen presented by the target cells. Of course, upon immune stimulation, B cells expressing a BCR capable of interacting with the target antigen may be subject to somatic hypermutation and/or may clonally expand. Hence, multiple B cells comprised in the B cells obtained from the subject may express the same B cell receptor, to which also may be referred as B cell clones.

The contacting step is such that the B cells will have sufficient time to interact with target cells and can form a bond. Such bonds may include bonds formed between the B cell receptor (BCR) as expressed by a B cell and the antigen presented by the cell. Such bonds, which may also be referred to as a specific bond, may include the formation of a synapse (Tolar and Spillane, Advances in Immunology, Chapter Three (2014, Vol. 123, pp 69-100). Cellular avidity relates to the strength of the bond between a B cell and a target cell, which is understood to be stronger when interaction is specific as opposed to non-specific interactions. A B cell-target cell interaction may not always result in a cell-cell bond. The contacting step is such that cell-cell bonds may be formed between B cells and target cells and appropriate conditions therefore are selected. It is understood that because the conditions are selected such that a specific interaction between the BCR of a B cell and said defined target antigen can occur, this necessarily implies that cell-cell bonds may be formed at the same time that do not involve the interaction between the BCR expressed by the B cell and the target antigen, or which involves a BCR—target cell interaction not specific for the target antigen.

“Cellular avidity” as used throughout herein relates to the overall strength of interactions occurring in a cell to cell contact, involving a diversity of molecules at the surfaces of the cells that interact. Such interactions may include a diversity of receptor-ligand pairs, among which e.g. a specific B cell receptor-target antigen interaction, occurring at the membrane surfaces of the cells. For example, when a B cell receptor recognizes an antigen presented at a target cell, bond formation may involve a multitude of interactions, as also other membrane bound molecules are involved in the interactions (such as integrins and the like), ultimately resulting in the formation of an immune synapse. Hence, “cellular avidity” may not be restricted to the interaction of e.g. the heavy and light chains of the BCR and the antigen expressed by the target cell, but rather involves a multitude of interactions working jointly forming a strong and/or specific bond between e.g. cells. It may also involve active signalling and processes internal to the cells such as e.g. during immune synapse formation. It is understood that the cellular avidity of B cells or of a B cell is defined relative to its target cell and may be dependent on assay conditions.

Once the B cells have had contact with the target cells, and have had the opportunity, if possible, to e.g. form a specific bond and/or a synapse, in a subsequent step a force is applied away from the target cells attached to the surface, such that at least part of the B cells bound to the target cells attached to the surface, and unbound B cells as well, move away from the cells attached to the surface. This way, target cells are obtained with B cells bound thereto, which are attached to the surface, wherein the exerted force was not sufficient to break cell-cell bonds formed. Of course, cells that are attached to the surface, to which no subsequent cells are bound, remain substantially attached as well. The B cells that remain bound after exerting the force representing to a larger extent, and thus are enriched for, B cells carrying a BCR capable of specifically interacting with the target antigen presented by the target cell. Hence, the means and methods in accordance with the invention which allow for manipulation of B cells, allow for enrichment, purification or selection of B cells that are capable of specifically interacting with an antigen.

As outlined herein, in the means and methods in accordance with the invention, it is preferred to have the target cells expressing the antigen of interest attached to the surface. Of course, it may be contemplated in an alternative embodiment to have the B cells attached to the surface. When performing the methods in accordance with the invention in this way, after the force has been exerted, this does off course not result in an enrichment or selection of B cells. Nevertheless, the percentage of target cells remaining bound after exerting a force still provides for valuable information indicative of the portion of B cells capable of binding with the target cells. Furthermore, one can contemplate to include further steps in the method to allow selection of B cells that remain bound to target cells after the force was exerted, e.g. via photo-activatable markers with which the B cells bound to target cells can be labelled. Alternatively, B cells bound with target cells may be subsequently separated from B cells not bound with target cells. For example, when target cells are labelled and/or after resuspension, pairs of B cells bound to target cells are separated from single B cells e.g. using flow-cytometry or micromanipulation. Hence, having B cells bound to the surface instead of the target cells expressing the antigen may still allow to manipulate B cells in accordance with the invention, though it may be more cumbersome. Hence, it is preferred to have the target cells attached to the surface.

As outlined herein, the invention as described herein is for example useful in means and methods for antibody discovery. Any method for antibody discovery may advantageously include manipulation, e.g. an enrichment, purification or selection, step as described herein utilizing cellular avidity between a B cell expressing a BCR and target cells expressing an antigen of interest. Hence, in one embodiment, a method of antibody discovery is provided, wherein the method comprises providing B cells expressing B cell receptors, wherein the B cells are allowed to interact with target cells expressing an antigen of interest, and wherein subsequently a differential force is exerted on the cells that have interacted, such that specifically bound B cells largely retain bound to target cells, whereas B cells bound less strongly, or not bound to target cells, become detached therefrom. This way, a biological highly relevant population of B cells can be provided that is highly useful in antibody discovery.

Thus, the steps of the methods as described herein allow for manipulation of B cells that are capable of specifically interacting with an antigen as presented by a cell via the BCR expressed by a B cell. Such manipulation is said highly advantageous for enrichment of B cells. Such manipulation also allows to obtain B cells which can subsequently be used for manufacturing e.g. of antibodies that are specific to a defined antigen. Moreover, the means and methods in accordance with the invention also allow to identify variable regions of the BCR and/or ranking thereof. In particular, the CDR regions of the variable regions of the BCR are of interest and may suffice, e.g. to identify sequences useful for the generation of antibodies. It is understood that the B cells provided as described herein may be from any subject, including naïve subjects. Of course, B cells provided from a subject exposed to the antigen may be preferred.

Hence, in one embodiment, a method is provided for enrichment of B cells expressing antigen-specific BCRs comprising the steps of:
a) providing B cells obtained from a subject,
b) providing target cells expressing the antigen, attached to a surface,
c) contacting the B cells with the target cells attached to a surface to allow the B cells to interact with the target cells,
d) exerting a force on the B cells, wherein the force is in a direction away from the target cells, and
e) removing unbound B cells and/or collecting the B cells that remained attached to the target cells.

In another embodiment, a method is provided for enrichment of B cells expressing antigen-specific BCRs comprising the steps of:
a) providing B cells obtained from a subject exposed to an antigen,
b) providing target cells expressing the antigen, attached to a surface,
c) contacting the B cells with the target cells attached to a surface to allow the B cells to interact with the target cells,
d) exerting a force on the B cells, wherein the force is in a direction away from the target cells, and
e) removing unbound B cells and/or collecting the B cells that remained attached to the target cells.

In an further embodiment, a method is provided for enrichment of B cells expressing antigen-specific BCRs comprising the steps of:
a) providing B cells obtained from a naïve subject,
b) providing target cells expressing the antigen, attached to a surface,
c) contacting the B cells with the target cells attached to a surface to allow the B cells to interact with the target cells,
d) exerting a force on the B cells, wherein the force is in a direction away from the target cells, and
e) removing unbound B cells and/or collecting the B cells that remained attached to the target cells.

It is understood that the last step of the method wherein unbound B cells are removed and/or the B cells that remained attached to the target cells are collected, results in an enrichment of B cells that express antigen-specific BCRs. It is understood that a B cell that expresses a BCR may also be referred to as a B cell expressing a membrane bound antibody, although the antibodies may not be secreted such as from plasma cells that develop from B cells. The B cells that remained attached to the target cells may be collected via methods known in the art. For example, cells may be trypsinized or the like or cells may be resuspended via mechanical means, wherein the B cells may remain bound to the target cells (obtaining doublets) and/or become unbound (obtaining single B cells).

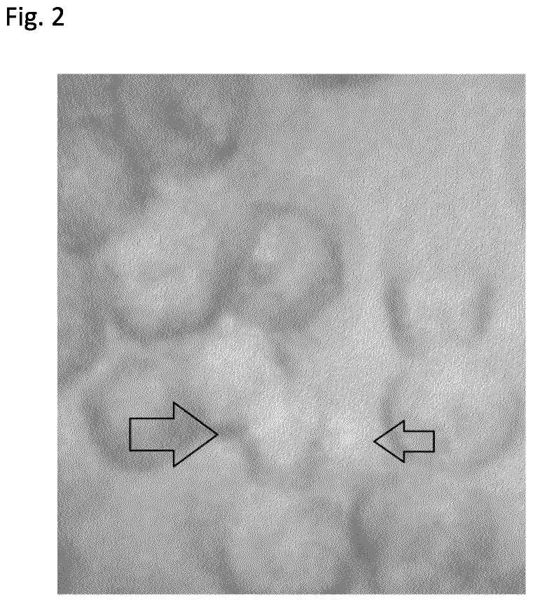

It is understood that the B cells that have remained attached to the target cells, may be further enriched. Hence, in a further embodiment in accordance with the invention, the B cells that remained attached to the target cells after the step of exerting the force are collected and further enriched. Suitable enrichment steps include e.g. FACS (fluorescence activated cell sorting) or MACS (magnetic activated cell sorting). For example, as shown in the example section, B cells bound to target cells after exerting the force may subsequently be identified with a (labelled) antigen or the like (see FIG. 2). The B cells may be contacted with the (labelled) antigen while attached to the cell surface (via the target cells), as shown in the example section after which the labelled B cells may be further collected and, optionally, enriched based on antigen binding. The B cells may also be collected first and subsequently bound with the antigen, after which a suitable enrichment step may be performed. For example, one may use e.g. FACS or MACS as enrichment methods utilizing a labelled antigen or an antigen bound to a magnetic bead.

The labelled antigen does not necessarily need to be presented in the same way as the antigen to which the subject has been exposed to (or suspected thereof) or as it is expressed by the target cells. It understood that antigens may be presented in any suitable form. Of course, the target cells expressing the antigen present it at their cell surface, or at least the extracellular part of the antigen, like or the same as presented in nature. For example, as shown in the examples, subjects were (suspected with a high degree of certainty of being) exposed to Sars-COV-2 (and/or a Sars-COV-2 vaccine), and B cells interacted with cells expressing Covid-19 spike protein, and subsequently B cells were stained with a labelled solubilized spike protein. Obviously, said antigens all comprise part of the same protein, i.e. the spike protein. Antigens are thus identical in amino acid sequence with regard to a substantial portion thereof. Hence, it is understood that the antigens are to have the same origin, and are identical at least in part and may have modifications and the like to make the antigens appropriate for the step of the method in which it is to be applied.

Hence, it is understood that the subsequent step may be performed with the same antigen. However, the antigen presented by the cells may be a relatively weak binding antigen in the soluble and/or conjugated state. By providing a modified version of it, e.g. in FACS or MACS methods and the like, the antigen may bind stronger which is useful for identifying cells, but which binding is not necessarily relevant from a biological perspective. From a biological perspective, it is more of interest to identify BCRs that can effectively bind to the antigen in its native state, i.e. as presented to the immune system in the (human) body. Nevertheless, MACS or FACS or the like may be contemplated to be included in the methods of the invention as additional steps. Thus, in the means and methods in accordance with the invention it may be contemplated to provide a labelled antigen, or the like, in order to stain the B cells that remain attached to the target cells to identify B cells that remain bound to the target cells after the force has been exerted.

Advantageously, it can also be contemplated to provide a further antigen. For example, one may use a B cell antigen. For example, CD27. One may provide different B cell antigens of interest with which different subpopulations of B cells may be identified. For example, memory B cells are of particular interest and one may select appropriate markers and/or enrichment methods to select these cells.

Furthermore, one may also provide for multiple antigens. As shown in the example section, cells were subsequently labelled with the spike variant D614. It may be contemplated to label the cells with further variants of the spike protein. For example, of the spike protein, variants are known, e.g. beta, delta, omicron etc., as well as lineages thereof. This way, B cells may be enriched for and/or identified that express a BCR that may recognize multiple highly related antigens. Such B cells may be of interest for identifying antibodies that can effectively bind to different and/or multiple variants of antigens. Conversely, one may identify B cells that are highly specific for a specific defined antigen. Hence, it is understood that the antigen used, e.g. in FACS or MACS or the like, or to the target cell, may not necessarily be the same as the antigen to which the subject was exposed, but can be highly related thereto. Hence, the antigen provided to the target cells or utilizing MACS or FACS may be the same or different, i.e. representing a variant of the antigen, e.g. based on observed variations in nature or the like. As long as the antigens are suitable for representing an antigen derived or related from the protein to which the subject has been exposed to (or suspected to have been exposed to), such antigens may be contemplated. Likewise, in a further embodiment, it may also be contemplated to provide for example different target cells with different antigens representing natural variants of the antigen to identify e.g. BCRs that are capable of specifically binding with a substantial or all natural variants, or which are capable of binding with a specific natural variant.

Hence, in one embodiment, a method is provided for identifying B cells expressing an antigen-specific BCR comprising the steps of:
a) providing B cells obtained from a subject exposed to an antigen,
b) providing target cells expressing the antigen, attached to a surface,
c) contacting the B cells with the target cells attached to a surface to allow the B cells to interact with the target cells,
d) exerting a force on the B cells, wherein the force is in a direction away from the target cells;
e) providing a labelled antigen,
f) staining the B cells that remained attached to the target cells with a labelled antigen, therewith identifying remaining B cells expressing antigen-specific B cell receptors, and
g) optionally, collecting the identified B cells expressing antigen-specific antibodies; and
wherein, optionally, one or more (natural) variants of said antigen are provided as labelled antigens and the B cells stained therewith.

The different labelled antigens may be differentially labelled. This way, B cells having a singly labelled cell may represent e.g. a highly specific BCR, and B cells having multiple labels, or all of the different labels provided, may represent a BCR which may be more broadly applicable. Likewise, in a further or different embodiment, it may also be contemplated to provide for example different target cells with different antigens representing natural variants of the antigen to identify e.g. BCRs that are capable of specifically binding with a substantial or all natural variants, or which are capable of binding with a specific natural variant.

In any case, whichever steps are applied in the methods of the invention, once B cells are collected which have interacted with the target cells and remained bound after the force was exerted, these B cells thus provided can be further advantageously used. For example, the collected B cells may be further cultured. The collected B cells may be immortalized. The collected B cells may be further cultured and immortalized. For example, the collected B cells may be further immortalized and cultured. Hence, the collected B cells may be further cultured and/or immortalized. Collected memory B cells may for example be induced into antibody producing plasma cells (see e.g. Jourdan et al. Blood, 2009 December; 114(25): 5173-5181) and subsequently be immortalized to provide for stable antibody producing cells. Means and methods for culturing B cells and/or immortalization of B cells are known in the art. For example, immortalization of B cells induced into plasma producing cells can be done by generating hybridoma and/or with EBV-transformation. This way, from the selected memory B cells, antibody producing cells may be generated and immortalized clones selected, and antibodies isolated therefrom, which are useful e.g. for antibody manufacturing and/or analysis and use thereof. In one embodiment, collected B cells are subsequently cultured and/or immortalized. In another embodiment, collected memory B cells are optionally differentiated into plasma B cells and immortalized. In a further embodiment, clones of immortalized cells are selected, e.g. for production of antibodies or the like.

Hence, a method is provided for providing B cells expressing an antigen-specific BCR comprising the steps of:
a) providing B cells obtained from a subject, preferably exposed to an antigen,
b) providing target cells expressing the antigen, attached to a surface,
c) contacting the B cells with the target cells attached to a surface, to allow the B cells to interact with the target cells,
d) exerting a force on the B cells, wherein the force is in a direction away from the target cells, e) removing unbound B cells and/or collecting the B cells that remained attached to the target cells,
f) optionally, further enriching collected B cells, e.g. with a labelled antigen,
g) immortalizing said collected B cells provided in step e) or as provided further enriched in step f),
h) selecting one or more B cell clones from the immortalized B cells, and
i) producing antibody from said one or more selected B cell clones.

In another embodiment, the invention provides for an enriched B cell population obtained or obtainable by any of the methods as described above. In a further embodiment, the enriched B cell population, which may be immortalized or not, and which may be memory B cells induced into plasma B cells, may be used to produce antibodies. Antibodies thus produced may be referred to as polyclonal antibodies. Of course, one may select clones therefrom to produce monoclonal antibodies. Hence, in a further embodiment, the collected B cells in accordance with the invention, which are preferably memory B cells, are allowed, for example by induction into plasma cells, to produce and secrete antibodies and obtaining said produced antibody and optionally preparing a pharmaceutical composition comprising the antibodies. In another embodiment, the collected B cells in accordance with the invention, which are preferably memory B cells, are allowed, for example by induction into plasma cells, and preferably immortalized, to produce and secrete antibodies and, a cell clone is selected therefrom, and subsequently from said cell clone antibody produced is obtained therefrom and optionally a pharmaceutical composition prepared comprising the antibody produced.

Of course, it may not be necessary to use the collected B cells to produce antibodies or the like. One may also simply sequence the variable regions of the BCR receptor sequences to obtain useful information representing the BCRs that were advantageously selected with the means and methods in accordance with the invention. Hence, in another embodiment, the sequences of the variable regions of the heavy chain and/or light chain of the antibodies expressed by B cells are determined. Sequencing may include genomic DNA sequencing or RNA sequencing. As B cells may be obtained from e.g. animals having common heavy or light chains (i.e. all B cells carry the same light chain or heavy chain sequence), it may suffice in such a scenario to only sequence the variable sequences, as the common chain is already known a priori. However, when both heavy and light chain of the BCR can vary, it is of interest to sequence both variable regions, i.e. the CDR regions, of both chains. Hence, single cell sequencing methods are highly useful to determine the sequences of both heavy and light chain variable regions (or simply the CDR regions) to identify sequences of interest. Single-cell sequencing for pairing of variable regions may thus be performed. Single-cell library preparation methods are generally more expensive and complex but offer several advantages, including i.a. simple counting of clonal fractions as cells are individually barcoded, and chain pairing as separate chains form the same cell can be identified. In one implementation, single-cell libraries may be prepared by dispensing individual B cells from a collected fraction into well plates by FACS or other single-cell dispensing methods and preparing full length RNA sequencing libraries using established protocols (e.g. Smart-seq3). These libraries can be sequenced directly or further enriched by targeted amplification using specific PCR primers for receptor sequences. Similar libraries may also be prepared using droplet based single-cell RNA sequencing methods (e.g. 10× genomics or InDrop). After sequencing receptor pairs, clonal cell numbers and other genes of interest can be bioinformatically quantified using the single-cell barcodes and UMIs incorporated during the library preparation.

Sequencing the variable region(s) and identifying unique sequences therefrom and quantifying these for unique sequences (i.e. a unique variable region (with a known common variable region) or unique variable region paired sequences) allows to identify cell clones and allows quantification e.g. of B cell numbers or the like as well. This way, a B cell expressing an antigen-specific BCR can be identified (or at least an antigen-specific BCR candidate is identified, which can e.g. be confirmed by producing an antibody and testing its specificity). Of course, the method may result in destruction of the B cell per se, but by identifying the sequence of the variable region(s) of the BCR, one identifies a B cell that was collected. Nevertheless, if B cells were collected and/or clonally expanded (e.g. by culturing and/or immortalization as described above), one may also subject a sample of such B cells to the sequencing methods to allow for B cells remaining for further use.

Hence in another embodiment, a method for identifying a B cell expressing an antigen-specific BCR comprising the steps of:
a) providing B cells obtained from a subject exposed to an antigen,
b) providing target cells expressing an antigen attached to a surface,
c) contacting the B cells with the target cells attached to a surface to allow the B cells to interact with the target cells,
d) exerting a force on the B cells, wherein the force is in a direction away from the target cells,
e) collecting the B cells that remained attached to the target cells and/or that detached from the target cells,
f) determining the sequence of the variable region of the heavy chain and/or the light chain expressed by the collected B cells and, optionally, of the provided B cells,
g) using the determined sequences to identify B cell clones and determine the number of B cells representing a B cell clone of the B cells that remained attached to the target cells and/or detached from the target cells, and, optionally, the number of B cells representing a B cell clone in the sample, and
h) identifying a B cell expressing an antigen-specific BCR based on the determined numbers by identifying B cell clones that are enriched in the fraction representing B cells attached to the target cells.

It is understood that in step g) of at least two populations, i.e. the sample, B cells that remain attached and B cells that detached from the target cells, a representative number of B cells having the sequences needs to be determined to allow for identifying cell clones enriched in the fraction representing B cells attached to the target cells. Hence, sequencing may be performed at any stage of the method of at least two of such populations.

In one embodiment, a method is provided for identifying B cells expressing an antigen-specific BCR comprising the steps of:
a) providing B cells obtained from a subject exposed to an antigen,
b) optionally, determining of a sample of the B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells and/or that detached from the target cells,
g) determining the sequence of the variable region of the heavy chain and/or the light chain expressed by the collected B cells,
h) using the determined sequences to identify B cell clones and determine the number of B cells representing a B cell clone of the B cells that remained attached to the target cells and/or detached from the target cells, and, optionally, the number of B cells representing a B cell clone in the sample,
i) identifying a B cell expressing an antigen-specific BCR based on the determined numbers by identifying B cell clones that are enriched in the fraction representing B cells attached to the target cells.

In another embodiment, a method is provided for identifying B cells expressing an antigen-specific BCR comprising the steps of:
a) providing B cells obtained from a subject,
b) optionally, determining of a sample of the B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells and/or that detached from the target cells,
g) determining the sequence of the variable region of the heavy chain and/or the light chain expressed by the collected B cells,
h) using the determined sequences to identify B cell clones and determine the number of B cells representing a B cell clone of the B cells that remained attached to the target cells and/or detached from the target cells, and, optionally, the number of B cells representing a B cell clone in the sample, and
i) identifying a B cell expressing an antigen-specific BCR based on the determined numbers by identifying B cell clones that are enriched in the fraction representing B cells attached to the target cells.

It is understood that of at least two populations provided in step b) and collected in step f) of the methods above, the sequences need to be determined to allow for identifying cell clones enriched in the fraction representing B cells attached to the target cells.

In any case, in these methods in accordance with the invention preferably, the number of cells representing a B cell clone is determined by quantifying nucleic acid sequences representing a unique cell clone. Alternatively, other means and methods aimed at identifying and quantifying the unique amino acid sequence as encoded by the receptors in cell clones may be contemplated. The numbers obtained can be used to calculate abundancy (fraction or percentage comprised in the obtained fractions provided e.g. representing the provided B cells obtained from the subject, the collected B cells that remained attached after the force was exerted, and/or of the B cells that were obtained that were detached due to the exerted force). Based on the determined numbers, it can be determined which B cell sequences, representing B cell clones, were enriched using the methods in accordance with the invention. For example, in case the fraction of a B cell clone is increased when compared to the fraction of B cells detached from the target cells or when compared to the initial B cells obtained from the subject, one can identify a B cell, or a B cell receptor sequence, representing an antigen-specific BCR, as these are enriched in the fraction that remained bound to the target cells.

Of course, one can also calculate e.g. the ratio between the determined different numbers from the fractions in order to arrive at a cellular avidity score, as this may allow to rank B cells or BCR sequences thereof. Hence, in a further step of the method provided that sufficient sequence information is available (one needs to determine relative abundancy for B cell clones in at least 2 of the 3 fractions, i.e. 1) of the fraction representing B cell clones of the provided B cells obtained from the subject, 2) of the B cells that were obtained that were detached due to the exerted force, and 3) of the collected B cells that remained attached after the force was exerted. This information may allow to determine a cellular avidity score for a BCR representing a B cell clone.

For example, in order to determine the relative cellular avidity, it may be sufficient to determine only the relative abundance of identified B cell clones in the B cells obtained from the subject and in the detached or attached B cell fraction. One can calculate the relative cellular avidity by dividing the relative abundance of the attached B cell fraction (or alternatively 1 minus the relative abundance of the sorted detached B cell fraction) with the relative abundance in the initial provided B cells. The relative cellular avidity thus calculated is indicative of the relative enrichment, i.e. if the value exceeds 1, there is an enrichment of the sequences representing a particular B cell clone. For example, if the value is 2, there are twice as much B cell clones present in the attached B cell fraction as compared with the B cells obtained from the subject. One can also determine cellular avidity as defined as a percentage (or ratio) of the B cell clones that remained attached to the target cells after exerting the force then it may be of importance to determine the abundance of these cell types in the cell sample in relation to the total number of B cells that are allowed to incubate onto the attached target cells (i.e. in relation to the total number of cells that are effectively sorted in detached and attached fractions by applying the force). One can also deduce these numbers based on analysing both detached and attached fractions. Cellular avidity is of course dependent on the testing conditions. When the same testing conditions are used, the latter cellular avidity score representing the fraction or percentage remaining bound, as opposed to fold enrichment, may allow for comparing different cell clones from independent experiments and rank these accordingly. Hence, in one embodiment, the cellular avidity score is either a cellular avidity score which is relative (i.e. representing fold enrichment) or which is absolute (representing percentage or fraction remaining bound under the conditions tested). Numbers can be calculated by determining a ratio, or as percentages. Whichever cellular avidity is determined, i.e. either relative or absolute, this allows to rank identified B cell clones or determined B cell receptors, accordingly. This way, and as also shown in the example section, a qualitative parameter relating to B cell receptors is provided which is highly useful e.g. in selecting antibody sequences, and which may be useful in development of antibodies for further applications.

Thus, in another embodiment, a method is provided for determining a cellular avidity score of B cell clones with target cells expressing an antigen comprising the steps of:
a) providing B cells obtained from a subject exposed to an antigen,
b) optionally, determining of a sample of the provided B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells, and/or, collecting B cells that detached from the target cells,
g) determining the sequence of the variable region of the heavy chain and/or the light chain expressed by the collected B cells,
h) using the determined sequences to identify B cell clones and determine the number of B cells representing a B cell clone of the B cells that remained attached and/or detached from the target cells, and optionally, the number of B cells representing a B cell clone in the sample, and
i) assign a cellular avidity score to identified B cell clones based on the determined numbers.

In yet another embodiment, a method is provided for determining a cellular avidity score of B cell clones with target cells expressing an antigen comprising the steps of:
a) providing B cells obtained from a subject,
b) optionally, determining of a sample of the provided B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells, and/or, collecting B cells that detached from the target cells,
g) determining the sequence of the variable region of the heavy chain and/or the light chain expressed by the collected B cells,
h) using the determined sequences to identify B cell clones and determine the number of B cells representing a B cell clone of the B cells that remained attached and/or detached from the target cells, and optionally, the number of B cells representing a B cell clone in the sample, and
i) assign a cellular avidity score to identified B cell clones based on the determined numbers. It is understood that of at least two populations provided in step b) and collected in step
f) in the methods described above, the sequences need to be determined and quantified to identify cell clones enriched in the fraction representing B cells attached to the target cells.

Memory B cells may also have underwent affinity maturation against an antigen, e.g. when an antigen or related antigens have been encountered by the subject on more than one occasion. Hence, it may also be advantageous to group unique sequences or closely related sequences, e.g. as these may be suspected to originate from a B cell clone that has underwent somatic hypermutation resulting in sequences being different but highly related. Hence, when identifying B cell clones, this may be taken into account, also when e.g. ranking identified B cells or BCRs.

It is understood and has been indicated herein that the B cells as provided from a subject comprise a variety of B cells, i.e. representing a variety of B cell receptors, the said B cells obtained from a subject are heterogeneous, at least with regard to the sequence of the B cell receptor that is expressed. Hence, the B cells as provided herein as obtained from a subject are not cancer B cells such as a cell line which represents a B cell cancer clone obtained from a subject. As explained herein, e.g. memory B cells, which are comprised in the B cell population are formed when an antigen is encountered and which may clonally expand. Hence, B cells obtained from a subject comprise B cells, with B cells expressing a unique B cell receptor, and wherein multiple B cells may express the same unique B cell receptor sequence. Accordingly, in a further embodiment, the said B cells that are provided are a heterogeneous population of B cells.

In the means and methods in accordance with the invention involving determining sequences of B cell receptors, the B cells that remained attached can be stained with one or more labelled antigens. In a further embodiment, as said, the sequences of at least the heavy and/or light chain variable regions are determined of B cells stained with one or more labelled antigens.

The B cells as provided herein may be optionally tagged. It is understood that any means to tag B cells as provided herein may be contemplated. For example, one may contemplate to tag a B cell with a photoactive label, or with a photoactivatable label. A photoactive label may be a dye that can be easily identified with means such as e.g. (fluorescence) microscopy or FACS. A photoactivatable label may also be provided that may be subsequently activated by illumination with light of a suitable wavelength only in a well-defined interaction region of the device (e.g. in a flow channel or in a defined region of a cell chamber) to photoactivate and/or switch the dye in order to tag the B cells that remained attached to the target cells after the force was applied. One can of course also use specific labels that identify specific B cell markers. This way, one may identify in particular e.g. memory B cells, which are B cells of particular interest as these express BCRs which may specifically bind with the antigen as presented by the target cell. Suitable B cell markers may include e.g. CD19, CD20, CD27, CD38, and CD24. Further suitable B cell markers include IgM, IgD, IgG, IgA. IgE (the cell surface BCRs), CD10, CD21, CD22, CD52, and CD267.

In particular of interest are as said, memory B cells. Hence, in a further embodiment, highly preferably, the B cells as provided from the subject are memory B cells. Memory B cells can be identified by the presence of the CD27 marker. For example, in humans, memory B cells are commonly identified by expression of CD27, coupled with low level expression of CD23/Fc epsilon RI, and lack of expression of the plasma cell marker, Syndecan-1/CD138. As shown in the example section, memory B cells may also be selected by including negative markers, e.g. a plasma cell marker, in order to obtain memory B cells, e.g. when B cells are isolated from the blood.

B cells can be further obtained from bone marrow, spleen, lymph nodes or tonsils, e.g. via a biopsy. These harbour memory B cells, especially shortly after being exposed to said antigen, e.g. after vaccination or infection. B cells as provided from a subject and as described herein throughout, may preferably be selected from the blood. This is because blood may be relatively easily and less invasively be obtained from subjects, and because blood comprises B cells that express B cell receptors such as memory B cells, which thus represents a valuable source of B cells highly useful in the means and methods in accordance with the invention.

In another embodiment, the B cells as provided in accordance with the means and methods in accordance with the invention are obtained from human. Domesticated animals, such as cats and dogs may be contemplated. Poultry, horses, cows, camels, and pigs may be contemplated as well. Any subject having B cells such as e.g. llamas, camels, sharks, monkeys, rats, mice, rabbits, chicken, platypus, and the like, may be a suitable source of B cells in accordance with the invention. Human B cells, or B cells expressing human (variable region) heavy and light chain sequences are in particular of interest for the development of antibodies suitable for human use. Hence, rats or mice provided with a human immune system (HIS), which are known in the art may be useful in that context, as well as e.g. animals provided with humanized heavy and light chain regions such the Memo mouse (from Merus), Velocimmune mouse (from Regeneron), or Kymouse (from Kymab). Hence, in one embodiment, the B cells that are provided are human B cells or B cells obtained from a subject, such as a humanized animal, that express heavy and light chain variable regions of human origin.

With regard to the cellular avidity, it is understood that this relates to the strength of binding of B cells (or control or reference cells thereof) to the target cells. It is understood that where we refer to specific forces applied to cells, this may refer to average forces, e.g. such forces may not be fully homogeneous, for example over the contact surface as may be the case with acoustic forces and shear-flow forces (see e.g. Nguyen, A., Brandt, M., Muenker, T. M., & Betz, T. (2021). Lab on a Chip, 21(10), 1929-1947 for a description of force inhomogeneities in acoustic force application). As is clear from the above, the type of force that is to be applied in accordance with the invention is a force capable of breaking cell-cell bonds, i.e. the force exerted causes B cells and target cells bound to each other move away from each other to such an extent that a cell-cell bond may break or rupture.

As shown in the example, a force that can be applied when target cells are attached to a surface is an acoustic force, such as ultrasonic forces as applied e.g. in a device as available from LUMICKS®, wherein the force is away from attached cells (e.g. such as in the LUMICKS® z-Movi® Cell Avidity Analyzer, e.g. as used by Larson et al., Nature 604, 7906:1-8, Apr. 13, 2022). However, the means and methods in accordance with the invention when exerting a force away from e.g. target cells attached to a surface as described herein is not be restricted to the use of acoustic force. Other types of forces that can be applied away from attached cells, such as centrifugal (or other acceleration) forces or a shear flow force can be applied as well. With such forces, similar results as depicted in the graphs and tables as shown in the examples herein may be provided, relying on the same inherent properties of cellular avidity between B cells and target cells. As long as a force can be applied and controlled on e.g. B cells that bind/interact with target cells attached to a surface (or vice versa) such a force can be contemplated in accordance with the invention. Hence, in a further embodiment, in the means and methods in accordance with the invention, a force is applied away from the target cells, wherein the force is acoustic force, a shear flow or a centrifugal force.

For centrifugal forces, it is easier to ensure that the force applied is homogeneous across the whole interaction region, since such a force does not depend strongly on a location on a surface with respect to a force transducer and/or the wall of a flow channel or sample holder. Accordingly, in connection to the subject matter disclosed herein, means and methods exist which allows one to exert forces on cells attached to a surface and collect the cells, detached and/or attached cells, on which defined forces have been exerted.

It is understood that with regard to the force exerted, the exact forces experienced by cells may also depend on cell size and or other cell properties such as density and compressibility. The force may be a nominal force and not the true force experienced by target cells or B cells. E.g. it may be hard to precisely predict the average cell size, density, compressibility, etc. of the cells and the force may have been calculated based on theory alone or may have been calibrated using test particles with specific (preferably known) properties (see e.g. Kamsma, D., Creyghton, R., Sitters, G., Wuite, G. J. L., & Peterman, E. J. G. (2016), Tuning the Music: Acoustic Force Spectroscopy (AFS) 2.0. Methods, 105, 26-33). The force may be such a calculated or calibrated force expressed with units of N (e.g. pN) but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014). Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices such devices are suitable for the means and methods in accordance with the invention.

A differential force means that the force on one cell differs from the force on the other cell with regard to direction of the force and/or the magnitude of the force, resulting in a net force allowing to break cell-cell bonds if the differential force exceeds the binding force. Hence, in the means and methods in accordance with the invention as described herein, in the step of applying a force on the B cells away from the target cells (or vice versa), instead a differential force may be applied which does not require attachment of the target cells to a surface.

For example, when a target cell bound to an effector cell, a doublet, is forced through a nozzle, the closer to the throat of the nozzle the faster the flow. This means that the first cell to enter the nozzle is subjected to a stronger acceleration than the cell lagging behind and the cells experience a differential force resulting in a net force which can result in cell-cell bond rupture, provided the force is large enough. A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J. Histochem. Cytochem. 1979, 27(1), 635-641). Also, by properly tuning the forces in a flow-cytometer normally used to measure cell deformations, such as e.g. described in Otto, et al. Nat. Methods 12, 199-202 (2015) suitable forces can be applied. Tuning can be achieved e.g. by changing the nozzle size or geometry and/or the flow speeds used. Other suitable devices known in the art may include a vortex mixer, with which shear forces may be suitably applied as well. Accordingly, in one embodiment, the force applied involves a shear force.

In yet another further embodiment not requiring attachment of cells, the differential force applied is an ultrasonic force. It is understood, as outline above, that such ultrasonic forces are not forces such as applied e.g. in a device as available from LUMICKS®, wherein the force is away from attached cells (e.g. such as in the LUMICKS® z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter II.2.8, page 576; and Moore et al., Experimental Cell Research, Volume 65, Issue 1, 1971, pages 228-232).

Accordingly, it is understood that in some embodiments, the differential force to be applied does not require either of the target cells or B cells, e.g. memory B cells, to be or remain to be attached, and the differential force may be a force selected from the range 1 pN-10 nN. In another embodiment, in methods in accordance with the invention, wherein the force that is applied is a differential force, neither the target cells nor the B cells, e.g. memory B cells, are required to be attached to a surface, and the differential force is applied in the range of 1 pN-10 nN, thereby providing cells substantially comprised of target cells, B cells, and B cells bound to target cells. Of course, when cells are not required to be attached, it is understood that cells may be differentially labelled, before and/or after the differential force is exerted in order to identify B cells that have remained attached after the force has been exerted and differentiate these from B cells that have detached from the target cells. Yet, having the target cells attached to a surface is a convenient way allowing to differentiate between detached B cells and B cells that remained attached, and at the same time allow for a convenient separation of these cells. Hence, the (differential force) to be applied advantageously requires either of the target cells or B cells, e.g. memory B cells, to be attached, and the force applied is directed away from the attached cells, and the (differential) force may be a force selected from the range of 1 pN-10 nN. It is understood that by selecting an appropriate force, aspecific cell-cell bonds may be (more) selectively broken, while retaining specific cell-cell bonds between B cells and target cells. For example, in case of B cells, B cells expressing a BCR which specifically interacted with a target antigen expressed by a target cells may be retained.

In any case, suitable applied forces which are known in the art include e.g. a force in the range of 1 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 1 pN, or at least 10 pN, or at least 20 pN, or at least 50 pN, or at least 100 pN, or at least least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. In another embodiment, the force that is applied is in the range of 10 pN-1 nN. For example, a suitable amount of force that can be exerted between cells (e.g. such as in the z-Movi® device) can be selected to be in the range of 200 pN-3000 pN. Of course, these force ranges are known to be useful with cells attached to a surface, and the maximum force that may be selected may exceed 3000 pN as it may not be required to have the cells to remain attached to a surface in accordance with the invention.

Hence, in view of the above, in further embodiments in the step of applying a force on the B cells away from the target cells, instead a differential force is applied which does not require attachment of the target cells to a surface and wherein said target cells are optionally attached to a surface.

Without being bound by theory, a specific interaction between a BCR of a B cell and a target antigen presented by a target cell can involve further surface molecules that can form an immunological synapse. As is understood the range of force that may break an aspecific cell-cell bond versus a specific cell-cell bond between a BCR on a B cell and a target antigen presented by a target cell, which may include synapse formation, differs. This difference can be to such an extent that the ranges of the required forces do not overlap. It is understood that some overlap may occur. Hence the force that is selected, as outline above, may allow for aspecific cell-cell bonds remaining and some specific cell-cell bonds that formed e.g. a synapse to break. In case there is substantially no overlap, a differential force can be selected, as outline above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed e.g. a synapse. In any case, whichever amount of differential and type of force selected, B cells that have had a specific interaction will largely remain as compared with B cells that did not have a specific interaction. This principle allows to enrich for, and identify, B cells expressing a BCR of interest useful in antibody discovery.

As said, the means and methods in accordance with the invention are in particular highly useful in discovery methods aimed at discovery of antibodies useful against antigens. Suitable antigens that can be contemplated in accordance with the invention may be selected from the group consisting of cancer antigens, allergens, antigens from any pathogen, including viruses, bacteria, fungi, and yeast, and donor antigens. In particular embodiment, a suitable antigen may be from coronavirus, e.g. from the spike protein.

As the invention allows to identify BCRs that represent antibodies, in a further embodiment, sequences of said identified BCRs thus provided may be used to provide for one or more nucleic acids encoding an antibody comprising the variable regions of the heavy and/or the light chain of the antigen-specific antibody. It is understood that any type of antibody may thus be encoded by said one or more nucleic acids, ranging from encoding antibodies in a format such as found in nature, e.g. in case of human antibodies consisting of dimerized full length heavy and light chain, up to engineered antibodies, such as bispecific antibodies, single chain antibodies, and antibodies with inert Fc regions or Fc regions deleted. Of course, sequences may also be utilized to express e.g. chimeric antigen receptors, such as CARs, which may be useful for transduction of effector cells for cell therapy.

Thus, in a further embodiment, said one or more nucleic acids encoding said antibody sequences may be provided to a cell to provide for a cell capable of producing an antibody comprising the variable regions of the heavy and/or light chain of the antigen-specific antibody. Such antibodies may be in particular useful, e.g. for diagnostic purposes. Such antibodies may also be useful for example for pharmaceutical compositions. Hence, in a further embodiment, the methods in accordance with the invention further comprise producing the antibody and obtaining said produced antibody and optionally preparing a pharmaceutical composition comprising the produced antibody.

Furthermore, antibodies thus produced with the means and methods of the invention as described herein are provided, or pharmaceutical compositions comprising said antibodies are provided as well.

In another embodiment of the present application, a method is provided of determining an immune response against an antigen comprising the steps of:
a) providing B cells, preferably memory B cells, obtained from a subject,
b) providing target cells expressing said antigen attached to a surface,
c) contacting the B cells with the target cells attached to a surface to allow the B cells to interact with the target cells,
d) exerting a force on the B cells, wherein the force is in a direction away from the target cells, and
e) determining the amount of B cells that remains attached to the target cells.

Of course, the methods in accordance with the invention are in particular useful for determining subjects exposed to an antigen, or subjects investigated of being exposed to said antigen, or subjects suspected of being exposed to said antigen. Hence, in another embodiment, a method is provided of determining an immune response against an antigen comprising the steps of:
a) providing B cells, preferably memory B cells, obtained from a subject exposed to an antigen or suspected or being investigated of being exposed to said antigen,
b) providing target cells expressing said antigen attached to a surface,
c) contacting the B cells with the target cells attached to a surface to allow the B cells to interact with the target cells,
d) exerting a force on the B cells, wherein the force is in a direction away from the target cells, and
e) determining the amount of B cells that remains attached to the target cells.

As said, the current invention now provides for means and methods that can be utilized to determine an immune response against an antigen, in particular by utilizing B cells obtained from a subject exposed to an antigen or suspected thereof, i.e. suspected of having been or being exposed to said antigen. When a subject, for example a human subject, has been or is exposed to an antigen, B cells normally develop in the subject which produce B cell receptors that can recognize the antigen. These B cells have the capacity to further develop into antibody producing cells. The subject will retain B cells, i.a. memory B cells, that express B cell receptors that can recognize the antigen, and which are capable of developing into antibody producing plasma B cells when the subject e.g. becomes again exposed to the antigen. Memory B cells may also have undergone affinity maturation against an antigen, e.g. when an antigen or related antigens have been encountered by the subject on more than one occasion. Thus, it is understood that B cells as provided from a subject comprise a variety of B cells, i.e. representing a variety of B cell receptors, and the said B cells obtained from a subject are heterogeneous, at least with regard to the sequence of the B cell receptor that is expressed. Hence, the B cells are not cancer B cells, such a cell line which represents a B cell cancer clone. As explained herein, e.g. memory B cells, which are comprised in the B cell population are formed when an antigen is encountered and which may clonally expand. Hence, B cells obtained from a subject comprise B cells, with each B cell expressing a unique B cell receptor, and wherein multiple B cells may express the same B cell receptor sequence.

Subjects can be exposed to an antigen via various means. A subject can be vaccinated, immunized, or infected. A subject may also have received a transplant, e.g. an organ transplant or cell therapy. For example, subjects can be exposed to a vaccine, or be naturally infected, in case of e.g. pathogens. A vaccine can comprise a live or dead pathogen. A vaccine preferably comprises a protein, e.g. from the pathogen, against which antibodies are to be induced or of an antigen of interest, e.g. a cancer antigen. A vaccine can be an attenuated virus, or a conventional vaccine, i.e. combining antigen or an inactivated pathogen with a suitable adjuvant. A vaccine can also be a DNA/RNA vaccine or based on a viral vector (e.g. adenovirus-based), capable of expressing the antigen. Pathogens can be algae, bacteria, fungi, prions, viroids, viruses, and parasites. It is understood that immunization or vaccination are terms that can be used interchangeable and mean the same, i.e. administering to a subject a vaccine which results in an immune response against an antigen provided by/with the vaccines. With regard to the subject, it is understood that these include vertebrates, for example mammals, that have a B-cell repertoire. For example, as described in the example section, B-cells can be obtained from subjects that have been, or are suspected to have been or being investigated therefor, exposed to antigen, either through natural infection and/or through vaccination.

Target cells expressing the antigen may be any cell presenting the antigen at its cell surface. For example, in case of a receptor which presents at the cell surface, a cell may be transfected or transduced with a vector construct to express said receptor. As described in the example section, a target cell may be e.g. a human cell line which e.g. attaches well to a surface. Of course, primary cells may be also be used. Cells may also be selected that present said antigen naturally, i.e. cells are not engineered. Suitable cells may include eukaryotic cells or prokaryotic cells. Cells that may be selected include fungal cells, animal cells, insect cells, mammalian cells, bacterial cells, yeasts, and protozoa. It may be preferred to select human cells as human subjects may be of particular interest in accordance with the invention.

As said, the cells, which express the antigen, can be attached to a surface. It is understood that surfaces for attaching cells may be any surface suitable for attaching cells. Suitable surfaces for attaching cells include plastic or glass surfaces. These surfaces may be coated e.g. with a protein to attach cells to the surface, such as poly-L-lysine or the like. Suitable polypeptides for attachment of target cells that may be contemplated and are known in the art include for example fibronectin, poly-L-lysine, poly-D-lysine, poly-L-ornithine, laminin, collagen, fibronectin, fibrinogen, vitronectin, or osteopontin. In any case, a suitable polypeptide or other suitable coating may be selected such that the target cells that are attached to the surface allow for the target cells to remain attached to the surface when applying a force on the heterogeneous population of cells. In other words, when a force is applied on these cells which allows for detachment of (some) of these cells from the target cells, the target cells are to substantially remain attached to the surface.

Next, the B cells are contacted with the target cells attached to a surface to allow the B cells to interact with the target cells. It is understood that the B cells, e.g. as obtained from a subject, are heterogeneous with regard to the BCR, i.e. each B cell expresses a defined B cell receptor and between B cells, B cells may express different B cell receptors. Different B cells expressing different BCRs are understood to be comprised in the provided B cells that are capable of interacting with the target antigen presented by the target cells. Of course, upon immune stimulation, B cells expressing a BCR capable of interacting with the target antigen may be subject to somatic hypermutation and/or may clonally expand. Hence, multiple B cells comprised in the B cells obtained from the subject may express the same B cell receptor, to which also may be referred as B cell clones.

The contacting step is such that the B cells will have sufficient time to interact with target cells and can form a bond. Such bonds may include bonds formed between the B cell receptor (BCR) as expressed by a B cell and the antigen presented by the cell. Such bonds, which may also be referred to as a specific bond, may include the formation of a synapse (Tolar and Spillane, Advances in Immunology, Chapter Three (2014), Vol. 123, pp 69-100). Cellular avidity relates to the strength of the bond between a B cell and a target cell, which is understood to be stronger when interaction is specific as opposed to non-specific interactions. A B cell-target cell interaction may not always result in a cell-cell bond. The contacting step is such that cell-cell bonds may be formed between B cells and target cells and appropriate conditions therefore are selected. It is understood that because the conditions are selected such that a specific interaction between the BCR of a B cell and said defined target antigen can occur, this necessarily implies that cell-cell bonds may be formed at the same time that do not involve the interaction between the BCR expressed by the B cell and the target antigen, or which involves a BCR-target cell interaction not specific for the target antigen.

“Cellular avidity” as used herein relates to the overall strength of interactions occurring in a cell to cell contact, involving a diversity of molecules at the surfaces of the cells that interact. Such interactions may include a diversity of receptor-ligand pairs, among which e.g. a specific B cell receptor-target antigen interaction, occurring at the membrane surfaces of the cells. For example, when a B-cell receptor recognizes an antigen presented at a target cell, bond formation may involves a multitude of interactions, as also other membrane bound molecules are involved in the interactions (such as integrins and the like), ultimately resulting in the formation of an immune synapse. Hence, “cellular avidity” may not be restricted to the interaction of e.g. the heavy and light chains of the BCR and the antigen expressed by the target cell, but rather involves a multitude of interactions working jointly forming a strong bond between e.g. cells. It may also involve active signalling and processes internal to the cells such as e.g. during immune synapse formation. It is understood that the cellular avidity of a B cell of a certain type is defined relative to its target cell and conditions tested.

Once the B cells have had contact with the target cells, and have had the opportunity, if possible, to e.g. form a specific bond and/or a synapse, in a subsequent step a force is applied away from the target cells attached to the surface, such that at least part of the B cells bound to the target cells attached to the surface, and unbound B cells as well, move away from the cells attached to the surface. This way, target cells are obtained with B cells bound thereto, which are attached to the surface, wherein the exerted force was not sufficient to break cell-cell bonds formed. Of course, cells that are attached to the surface, to which no subsequent cells are bound, remain substantially attached as well. The B cells that remain bound after exerting the force representing to a large extent or a substantial part, B cells carrying a BCR capable of specifically interacting with the target antigen presented by the target cell. This way, whether or not a substantial immune response has been triggered is determined, as the amount of B cells that remain attached is an indication thereof. For example, shortly after exposure, one may expect more B cells to attach, longer after exposure, the amount of B cells attached may represent a qualitative indication of successful long-term protection.

Determining an immune response can be performed e.g. in assessing whether or not a subject is infected with a pathogen. It can also be performed to assess whether or not a vaccination is effective. Likewise, in case of transplantation, e.g. of organs or cells of a donor to a subject, it can also be performed to assess whether or not the immune system of the subject (transplant recipient) mounts an immune response against the transplanted organ or cells, or, in case the transplant involves immune cells, including B cells, whether or not the transplanted B cells mounted an immune response against the subject. In any case, determining whether or not an immune response is occurring (or has occurred) against a defined antigen is highly important in the study and development of medicines, such as vaccines, as well as in various medical treatments and conditions. It is also understood that of course, in case of developing medicines aimed at immune suppression, the means and methods as described herein are likewise advantageous, as it allows one to assess with whether or not sufficient immune suppression can be obtained.

It is understood that in the method, after the force has been exerted, it may be contemplated to remove unbound B cells, resulting in B-cells remaining that express antigen-specific BCRs and which are capable of binding with the target cells and remaining bound thereto. It is understood that a B-cell that expresses a BCR may also be referred to as a B cell expressing a membrane bound antibody, although the antibodies may not be secreted such as from e.g. plasma cells that develop from B cells.

It is understood that determining the amount of B cells that remains attached with the target cells may also be regarded as providing a measure of cellular avidity. As of course the B cells provided of the subject, are substantially heterogeneous, i.e. having a variety of different B cell receptors, this means that the measure of cellular avidity as determined in this way, is a reflection of the cellular avidity from the different B cells provided combined. It is understood that the amount of B cells that remains attached may be determined using various means. For example, it may be sufficient to determine the amount of B cells in the detached fraction, and know the amount of B cells initially provided to interact with the target cells. It is highly preferred to determine the amount of B cells remaining attached relative to the amount of B cells initially provided, e.g. as a ratio, or as a percentage (such as e.g. shown in the examples). Such a ratio or percentage may be referred to as a cellular avidity score. Cellular avidity is of course dependent on the testing conditions. When the same testing conditions are used, the latter cellular avidity score representing the fraction or percentage remaining bound, may allow for comparing e.g. different subjects or different samples from a subject from independent experiments and rank these accordingly. This way, and as also shown in the example section, a qualitative parameter relating to the B cell population is provided which is highly useful e.g. in determining and comparing immune responses.

As outlined herein, in the means and methods in accordance with the invention, it is preferred to have the target cells expressing the antigen of interest attached to the surface. Of course, it may be contemplated in an alternative embodiment to have the B cells attached to the surface. Thus, in an further embodiment, methods for determining an immune response against an antigen in accordance with the invention comprise the steps of:
a) providing B cells, preferably memory B cells, obtained from a subject exposed to an antigen or suspected of being exposed to said antigen,
b) providing target cells expressing said antigen, wherein either the B cells or target cells are attached to a surface,
c) contacting the B cells with the target cells and allow the B cells to interact with the target cells,
d) exerting a force, wherein the force is in a direction away from the cells attached to the surface, and
e) determining the amount of B cells that remains attached to the target cells.

When performing the methods in accordance with the invention having the B cells attached to the surface, after the force has been exerted, determining the amount of B cells does off course not result in the amount of B cells that remain attached to target cells. Nevertheless, the number of target cells remaining bound to B cells after exerting a force may still provide for valuable information indicative of the portion of B cells capable of binding with the target cells. Furthermore, one can contemplate to include further steps in the method to allow selection or identification of B cells that remain bound to target cells after the force was exerted, e.g. via photo-activatable markers with which the B cells bound to target cells can be labelled. Hence, B cells bound with target cells may differentiated from B cells not bound with target cells. In any case, it may be contemplated to have the B cells bound to the surface instead of the target cells, as such a method still allows to determine the amount or relative amount of B cells that remains attached to the target cells, but it is preferred to have the target cells attached to the surface.

An antigen presented by the cells may be a relatively weak binding antigen when provided in the soluble and/or conjugated state. By providing a modified version of it, e.g. in FACS or MACS methods and the like, an antigen may bind stronger which is useful for identifying cells, but not necessarily relevant from a biological perspective. From a biological perspective, it is more of interest to have BCRs bind to the antigen in its native state, i.e. as presented to the immune system in the (human) body. Nevertheless, it may be contemplated to provide antigen, preferably a labelled antigen, or the like, in order to stain the B cells that remain attached to the target cells to identify B cells that remain bound to the target cells after the force has been exerted. In a further embodiment, at least the cells that remain bound after exerting the force are subsequently labelled with said antigen. As shown in the examples, B cells that remain bound after exerting a force can be subsequently labelled with the antigen. This way, B cells that remain bound and are capable of specifically binding to the antigen can be advantageously identified.

It is understood that the antigen does not necessarily need to be presented in the same way as the antigen to which the subject has been exposed (or suspected thereof) or as it is expressed by the target cells. It is understood that antigens may be presented in any suitable form. Of course, the target cells expressing the antigen highly preferably present the antigen, or at least the extracellular part thereof, as it is presented in nature. For example, as shown in the examples, subjects were (suspected with a high degree of certainty of being) exposed to Sars-COV-2 (and/or a SARS-COV-2 vaccine), and B cells interacted with cells expressing Covid-19 spike protein, and subsequently B cells were stained with a labelled solubilized spike protein. Obviously, said antigens all comprise part of the same protein, i.e. the spike protein. Antigens are thus identical in amino acid sequence with regard to a substantial portion thereof. Hence, it is understood that the antigens are to have the same origin, and are identical at least in part and may have modifications and the like to make the antigens appropriate for the step of the method in which it is to be applied.

In another further embodiment, the method of determining an immune response against an antigen as described herein comprises the further steps of:
f) providing a blocking antigen,
g) performing steps c) and d) with the B cells provided in step a), wherein the B cells are subjected to the blocking antigen provided in step f) prior to performing steps c) and d),
h) subsequently determining after step g) the amount of B cells that remains attached to the target cells, and
i) comparing the amount of B cells that remains attached in step e) without blocking antigen with the amount of B cells that remains attached in step h) with blocking antigen.

In this embodiment, in addition to the B cells interacting with the target cells, in a reference assay for comparison, B cells are first allowed to interact with a blocking antigen, prior to interacting with the target cells. It is understood that a blocking antigen is an antigen that blocks the interaction between a B cell and a (memory) B cell. For example, because the antigen binds well with the BCR and/or because it sterically hinders the interaction between the BCR and the antigen as presented by the target cell. This way, the amount of B cells that remain attached corresponds with the amount of B cells that had an aspecific interaction with the target cells. By comparing the amount of B cells that remain attached without blocking antigen with the amount of B cells with blocking antigen, the presence of a substantial immune response can be determined, as e.g. the difference between the amounts is an indicative of an immune response. If the difference is large, this can be indicative of a substantial immune response. If the difference is low, this is indicative of a poor, or lack of an, immune response.

As said, a reference may be provided by providing B cells of the subject that have been subjected to a blocking antigen. Of course, other references may be contemplated. Hence, in another embodiment, the methods in accordance with the invention comprise comparing the amount(s) of B cells that remain(s) attached with a reference. A reference can be e.g. B cells of a subject known to have mounted an immune response, or B cells of a subject known not to have mounted an immune response. It may be preferred to have a reference of a subject known not to have been exposed to said antigen, as immune responses may vary between individuals. Hence, in a further embodiment, said reference is provided by performing the method with B cells obtained from a control subject known not to be exposed to said antigen, and wherein the amount of B cells that remains attached is compared between the reference and the subject of interest. This way, when the amount of B cells is relatively high as compared with the reference, this is indicative of an immune response.

Said reference may be provided by performing the method of the invention with B cells obtained from a control subject known not to be exposed to said antigen, and wherein the amount of B cells that remains attached is determined and is compared between the control subject and the subject exposed to an antigen or suspected of being exposed to said antigen.

Of course, the reference may also be derived from the subject itself, e.g. as shown in the example section. Hence, in another embodiment, said reference is provided by obtaining B cells from a subject and separating said B cells into non-memory B cells and memory B cells, wherein the non-memory B cells are the reference for the memory B cells, and the method is performed separately with both memory B cells and non-memory B cells, and the amount of B cells that remains attached is compared between the reference non-memory B cells and memory B cells.

In any case, whichever suitable reference is used to compare the amount of B cells that remain bound thereto, the amounts are compared. It is understood that when conditions are the same (i.e. the same amount of B cells interacted with the same amount of target cells controlling interaction conditions to allow a comparison) the absolute amounts may be compared, e.g. the amount of B cells, or the percentage or ratio of B cells that remain bound (also referred to as cellular avidity score, in the methods herein for a heterogenic population of B cells). Comparing the amount of B cells may involve any type of comparison, such comparison results in cellular avidity index. It may be preferred, such as shown in the example section, to calculate the ratio or the difference between the amounts of B cells that remain attached. Such a ratio or difference may be referred to as a cellular avidity index, i.e. involving a comparison of a (relative) cellular avidity score relative to a suitable control. That ratio or difference may be determined between determined absolute amounts or percentages of B cells that remained bound, such as shown in the examples herein. In a further embodiment, the ratio or difference is calculated between: B cells and reference B cells; memory B cells and non-memory B cells; or memory B cells in non-blocking conditions and memory B cells in blocking conditions. In scenarios such as described in the examples, wherein ratios are calculated relative to the control and the ratio exceeds substantially 1, this may be indicative of an immune response against said antigen. Of course, the number calculated depends on the comparison made. If, e.g. a reference sample are B cells of a subject known to have mounted a highly efficient immune response, ratio's determined and thus cellular indices may be close to 1 when a subject has been exposed to an antigen. The ratio or difference determined may be indicative and a measure relating to the strength of the immune response, e.g. the higher the ratio, the stronger the immune response. Ranking cellular avidity indices may thus allow to compare different subjects and/or different testing conditions qualitatively.

In a further embodiment, the B cells that remained attached to the target cells may be stained with one or more antigens. For example, any suitable further antigen, other than the antigen of interest, i.e. against which an immune response is determined, may be contemplated. For example, antigens against which it is desirable to have cross reactivity against, or against which it is undesirable. By determining such further antigens, e.g. in addition to the antigen of interest, a measure of specificity may be provided. For example, in case the different antigens are provided with different labels, the reactivity towards related antigens can be determined as B cells will carry multiple BCRs and thus potentially may bind multiple (different) antigens. In another, or further embodiment, the method is performed with different target cells expressing different antigens. By performing the method with different target cells, e.g. expressing different antigens, e.g. from related antigens (such as in the case of Covid-19, from different variants of the spike protein, e.g. beta, delta, omicron etc., as well as lineages thereof), immune responses against these highly similar but different antigens can be determined. This may e.g. be advantageous to determine with which variant a subject has been infected. This may e.g. also be advantageous when developing broad neutralizing vaccines aimed at inducing an immune response against a large panel of variants. In any case, by performing the method with different target cells expressing different antigens, and/or staining with one or more labelled antigens, one can assess the immune response with regard to its specificity to said antigen.

The B cells as provided herein may be optionally tagged. It is understood that any means to tag B cells as provided herein may be contemplated. For example, one may contemplate to tag a B cell with a photoactive label, or with a photoactivatable label. A photoactive label may be a dye that can be easily identified with means such as e.g. (fluorescence) microscopy or FACS. A photoactivatable label may also be provided that may be subsequently activated by illumination with light of a suitable wavelength only in a well-defined interaction region of the device (e.g. in a flow channel or in a defined region of a cell chamber) to photoactivate and/or switch the dye in order to tag the B cells that remained attached to the target cells after the force was applied. One can of course also use specific labels that identify specific B cell markers. This way, one may identify in particular e.g. memory B cells, which are B cells of particular interest as these express BCRs which may specifically bind with the antigen as presented by the target cell. Suitable B cell markers may include e.g. CD19, CD20, CD27, CD38, and CD24. Further suitable B cell markers include IgM, IgD, IgG, IgA. IgE (the cell surface BCRs), CD10, CD21, CD22, CD52, and CD267.

As said, in particular of interest are memory B cells. Hence, in a further embodiment, highly preferably, the B cells as provided from the subject are memory B cells. Memory B cells can be identified by the presence of the CD27 marker. For example, in humans, memory B cells are commonly identified by expression of CD27, coupled with low level expression of CD23/Fc epsilon RI, and lack of expression of the plasma cell marker, Syndecan-1/CD138. As shown in the example section, memory B cells may also be selected by including negative markers, e.g. a plasma cell marker, in order to obtain memory B cells, e.g. when B cells are isolated from the blood.

In another embodiment, the B cells as provided in accordance with the means and methods in accordance with the invention are obtained from human. Of course, any subject having B cells, and of which it is of interest to determine an immune response, or immune status, with regard to an antigen may be contemplated. Domesticated animals, such as cats and dogs may be contemplated. Poultry, cows, horses, camels, and pigs may be contemplated as well. Any subject having B cells such as e.g. monkeys, sharks, llamas, camels, rabbits, rats, mice, chicken, platypus, and the like, may be a suitable source of B cells in accordance with the invention. Human B cells, or B cells expressing human (variable region) heavy and light chain sequences may be of particular interest as well. Hence, rats or mice provided with a human immune system (HIS), which are known in the art may be useful in that context, as well as e.g. animals provided with humanized heavy and light chain regions such the Memo mouse (from Merus), Velocimmune mouse (from Regeneron), or Kymouse (from Kymab). Hence, in one embodiment, the B cells that are provided are human B cells or B cells obtained from a subject, such as a humanized animal, that express heavy and light chain variable regions of human origin.

With regard to cellular avidity, it is understood that this relates to the strength of binding of B cells (or control or reference cells thereof) to the target cells. It is understood that where we refer to specific forces applied to cells this may refer to average forces, e.g. such forces may not be fully homogeneous, for example over the contact surface as may be the case with acoustic forces and shear-flow forces (see e.g. Nguyen, A., Brandt, M., Muenker, T. M., & Betz, T. (2021). Lab on a Chip, 21(10), 1929-1947 for a description of force inhomogeneities in acoustic force application). As is clear from the above, the type of force that is to be applied in accordance with the invention is a force capable of breaking cell-cell bonds, i.e. the force exerted causes B cells and target cells bound to each other move away from each other to such an extent that a cell-cell bond may break or rupture.

As shown in the example, a force that can be applied when target cells are attached to a surface is an acoustic force, such as ultrasonic forces as applied e.g. in a device as available from LUMICKS®, wherein the force is away from attached cells (e.g. such as in the LUMICKS® z-Movi® Cell Avidity Analyzer, e.g. as used by Larson et al., Nature 604, 7906:1-8, Apr. 13, 2022). However, the means and methods in accordance with the invention when exerting a force away from e.g. target cells attached to a surface as described herein is not be restricted to the use of acoustic force. Other types of forces that can be applied away from attached cells, such as centrifugal (or other acceleration) forces or a shear flow force can be applied as well. With such forces, similar results as depicted in the graphs and tables as shown in the examples herein may be provided, relying on the same inherent properties of cellular avidity between B cells and target cells. As long as a force can be applied and controlled on e.g. B cells that bind/interact with target cells attached to a surface (or vice versa) such a force can be contemplated in accordance with the invention. Hence, in a further embodiment, in the means and methods in accordance with the invention, a force is applied away from the target cells, wherein the force is acoustic force, a shear flow or a centrifugal force.

For centrifuge forces it is easier to ensure that the force applied is homogeneous across the whole interaction region since such a force does not depend strongly on a location on a surface with respect to a force transducer and/or the wall of a flow channel or sample holder. Accordingly, in connection to the subject matter disclosed herein, means and methods exist which allows one to exert forces on cells attached to a surface and collect the cells, detached and/or attached cells, on which defined forces have been exerted.

It is understood that with regard to the force exerted, the exact forces experienced by cells may also depend on cell size and or other cell properties such as density and compressibility. The force may be a nominal force and not the true force experienced by target cells or B cells. E.g. it may be hard to precisely predict the average cell size, density, compressibility, etc. of the cells and the force may have been calculated based on theory alone or may have been calibrated using test particles with specific (preferably known) properties (see e.g. Kamsma, D., Creyghton, R., Sitters, G., Wuite, G. J. L., & Peterman, E. J. G. (2016). Tuning the Music: Acoustic Force Spectroscopy (AFS) 2.0. Methods, 105, 26-33). The force may be such a calculated or calibrated force expressed with units of N (e.g. pN) but it may also be expressed without calibration as the input power (Vpp) applied to a piezo element (see Sitters, G., Kamsma, D., Thalhammer, G., Ritsch-Marte, M., Peterman, E. J. G., & Wuite, G. J. L. (2014). Acoustic force spectroscopy. Nature Methods, 12(1), 47-50), as angular velocity squared (ω²) in the case of centrifugal force application or as flow speed v and or as shear stress (Pa) in applications using shear forces. As long as the forces exerted by the devices, e.g. shear force, acoustic force, or centrifugal forces, but not limited thereto, can be varied and controlled and reproduced in such devices such devices are suitable for the means and methods in accordance with the invention.

A differential force means that the force on one cell differs from the force on the other cell with regard to direction of the force and/or the magnitude of the force, resulting in a net force allowing to break cell-cell bonds if the differential force exceeds the binding force. Hence, in the means and methods in accordance with the invention as described herein, in the step of applying a force on the B cells away from the target cells (or vice versa), instead a differential force may be applied which does not require attachment of the target cells to a surface.

For example, when a target cell bound to an effector cell, a doublet, is forced through a nozzle, the closer to the throat of the nozzle the faster the flow. This means that the first cell to enter the nozzle is subjected to a stronger acceleration than the cell lagging behind and the cells experience a differential force resulting in a net force which can result in cell-cell bond rupture, provided the force is large enough. A differential force that can be applied includes a shear force, e.g. such as can be applied utilizing repeated pipetting (repeated upwards and downwards flow of the sample) or flow through a nozzle.

Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J. Histochem. Cytochem. 1979, 27(1), 635-641). Also, by properly tuning the forces in a flow-cytometer normally used to measure cell deformations, such as e.g. described in Otto, et al. Nat. Methods 12, 199-202 (2015) suitable forces can be applied. Tuning can be achieved e.g. by changing the nozzle size or geometry and/or the flow speeds used. Other suitable devices known in the art may include a vortex mixer, with which shear forces may be suitably applied as well. Accordingly, in one embodiment, the force applied involves a shear force. In yet another further embodiment not requiring attachment of cells, the differential force applied is an ultrasonic force. It is understood, as outline above, that such ultrasonic forces are not forces such as applied e.g. in a device as available from LUMICKS®, wherein the force is away from attached cells (e.g. such as in the LUMICKS® z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter II.2.8, page 576; and Moore et al., Experimental Cell Research, Volume 65, Issue 1, 1971, Pages 228-232).

Accordingly, it is understood that in some embodiments, the differential force to be applied does not require either of the target cells or B cells, e.g. memory B cells, to be or remain to be attached, and the differential force may be a force selected from the range of 1 pN-10 nN. In another embodiment, in methods in accordance with the invention wherein the force that is applied is a differential force, neither the target cells nor the B cells, e.g. memory B cells, are required to be attached to a surface, and the differential force is applied in the range of 1 pN-10 nN, thereby providing cells substantially comprised of target cells, B cells, and B cells bound to target cells. Of course, when cells are not required to be attached, it is understood that cells may be differentially labelled, before and/or after the differential force is exerted in order to identify B cells that have remained attached after the force has been exerted and differentiate these from B cells that have detached from the target cells. Yet, having the target cells attached to a surface is a convenient way allowing to differentiate between detached B cells and B cells that remained attached, and at the same time allow for a convenient separation of these cells. Hence, the (differential force) to be applied advantageously requires either of the target cells or B cells, e.g. memory B cells, to be attached, and the force applied is directed away from the attached cells, and the (differential) force may be a force selected from the range of 1 pN-10 nN. It is understood that by selecting an appropriate force, aspecific cell-cell bonds may be (more) selectively broken, while retaining specific cell-cell bonds between B cells and target cells. For example, in case of B cells, B cells expressing a BCR which specifically interacted with a target antigen expressed by a target cells may be retained.

In any case, suitable applied forces which are known in the art include e.g. a force in the range of 1 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 1 pN, at least 10 pN, at least 20 pN, at least 50 pN, at least 100 pN, or at least least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 10 pN-10 nN, from 500 pN-10 nN, or from 1 nN-10 nN. In still a further embodiment, the force is selected from the range of 500 pN-5 nN, from 500 pN-4 pN, from 500 pN-3 pN. In one embodiment, the force that is applied is in the range of 1 pN-10 nN. In another embodiment, the force that is applied is in the range of 10 pN-10 nN. For example, a suitable amount of force that can be exerted between cells (e.g. such as in the z-Movi® device) can be selected to be in the range of 200 pN-3000 pN. Of course, these force ranges are known to be useful with cells attached to a surface, and the maximum force that may be selected may exceed 3000 pN as it may not be required to have the cells to remain attached to a surface in accordance with the invention.

Without being bound by theory, a specific interaction between a BCR of a B cell and a target antigen presented by a target cell can involve further surface molecules that can form an immunological synapse. As is understood the range of force that may break an aspecific cell-cell bond versus a specific cell-cell bond between a BCR on a B cell and a target antigen presented by a target cell, which may include synapse formation, differs. This difference can be to such an extent that the ranges of the required forces do not overlap. It is understood that some overlap may occur. Hence the force that is selected, as outline above, may allow for aspecific cell-cell bonds remaining and some specific cell-cell bonds that formed e.g. a synapse to break. In case there is substantially no overlap, a differential force can be selected, as outline above, which allows substantially for aspecific cell-cell bonds to break, while substantially retaining specific cell-cell bonds that formed a synapse. In case there is no overlap, and ranges are sufficiently far apart, a differential force may be selected, as outline above, which allows for aspecific cell-cell bonds to break while retaining specific cell-cell bonds that formed e.g. a synapse. In any case, whichever amount of differential and type of force selected, B cells that have had a specific interaction will largely remain as compared with B cells that did not have a specific interaction. This principle, based on cellular avidity, allows to differentiate between B cells obtained from subjects exposed and not exposed to antigens.

As said, the means and methods in accordance with the invention are in particular highly useful for determining immune responses against antigens. Suitable antigens that can be contemplated in accordance with the invention may be selected from the group consisting of cancer antigens, allergens, antigens from any pathogen, including viruses, bacteria, fungi, and yeast, and donor antigens. In a particular embodiment, a suitable antigen may be from coronavirus, e.g. from the spike protein.

The present application also describes a method of determining an immune response against an antigen comprising the steps of:
a) providing B cells, preferably memory B cells, obtained from a subject exposed to an antigen or suspected of being exposed to said antigen,
b) providing target cells expressing the antigen attached to a surface,
c) contacting the B cells with the target cells attached to a surface to allow the B cells to interact with the target cells,
d) exerting a force on the B cells, wherein the force is in a direction away from the target cells, and
e) determining the amount of B cells that remains attached to the target cells.

The present application also describes a method in accordance with the previously described method, wherein at least the cells that remain bound after exerting the force are subsequently labelled with said antigen.

The present application also describes a method of determining an immune response against an antigen, in accordance with any one of the previously described methods comprising the further steps of:
f) providing a blocking antigen,
g) performing steps c) and d) with the B cells provided in step a), wherein the B cells are subjected to the blocking antigen provided in step f) prior to performing steps c) and d),
h) subsequently determining after step g) the amount of B cells that remains attached to the target cells, and
i) comparing the amount of B cells that remains attached in step e) without blocking antigen with the amount of B cells that remains attached in step h) with blocking antigen.

The present application also describes a method in accordance with any one of the previously described methods comprising comparing amount(s) of B cells that remain(s) attached with a reference.

The present application also describes a method in accordance with the previously described method, wherein said reference is provided by performing the method with B cells obtained from a control subject known not to be exposed to said antigen, and wherein the amount of B cells that remains attached is determined and is compared between the control subject and the subject exposed to an antigen or suspected of being exposed to said antigen.

The present application also describes a method in accordance with the two previously described methods, wherein said reference is provided by obtaining B cells from a subject and separating said B cells into non-memory B cells and memory B cells, wherein the non-memory B cells are the reference for the memory B cells, and the method is performed separately with both memory B cells and non-memory B cells, and the amount of B cells that remains attached is compared between the reference non-memory B cells and the memory B cells.

The present application also describes a method in accordance with any one of the four previously described methods, wherein comparing comprises calculating a ratio or calculating a difference between the determined amounts of B cells that remains attached.

The present application also describes a method in accordance with the previously described method, wherein the ratio or difference is calculated between:
the amount of B cells and the amount of reference B cells,
the amount of memory B cells and the amount of non-memory B cells, or
the amount of memory B cells in non-blocking conditions and the amount of memory B cells in blocking conditions.

The present application also describes a method in accordance with any of the previously described methods, wherein the B cells that remained attached to the target cells after exerting the force are stained with one or more labelled antigens and/or wherein the method is performed with different target cells expressing different antigens and/or herein the B cells are tagged.

The present application also describes a method in accordance with any of the previously described methods, wherein the B cells comprise CD27+ B cells and/or wherein when the B cells provided are memory B cells, these B cells are CD27+, and wherein when B cells provided are non-memory B cells, these B cells are CD27−.

The present application also describes a method in accordance with any of the previously described methods, wherein the B cells are obtained from blood, and/or wherein the B cells are obtained from a human subject.

The present application also describes a method in accordance with any of the previously described methods, wherein the force that is applied is in the range of 1 pN-10 nN.

The present application also describes a method in accordance with any of the previously described methods, wherein the force is an acoustic force, a shear flow force or a centrifugal force.

The present application also describes a method in accordance with any of the previously described methods, wherein in the step of applying a force on the B cells away from the target cells, instead a differential force is applied which does not require attachment of the target cells to a surface and wherein said target cells are optionally attached to a surface.

The present application also describes a method in accordance with any of the previously described methods, wherein the antigen is from a cancer, a pathogen or a donor, or is an allergen.

### EXAMPLES

### Example 1

HEK293 cells expressing Covid-19 spike protein (InvivoGen, Cat #293-cov2-s) were cultured in DMEM media supplemented with 10% FBS (DMEM was obtained from Thermo Fisher, cat. #10567014 and FBS from Sigma-Aldrich, cat. #F2442). Cells were passaged (1:5 to 1:10) every 2 or 3 days after using standard cell culture methods for adherent cells.

The B cell populations were obtained from Sanquin from fresh human blood by using a MACS sorting technique. Sanquin provides blood products prepared from blood donors in the Netherlands. The prevalence in the Netherlands of adults which received vaccines and/or had been infected with Covid-19 was high (Sanquin indicated that in sept-dec 2021 they found antibodies against covid in 97% of their donors). The memory B cells and non-memory B cell populations were obtained using first negative selection and subsequent isolation of memory B cells therefrom with positive selection, with the non-memory B cell population remaining in solution after positive selection of the memory B cells (utilizing the Memory B cell Isolation KIT from Miltenyi Biotec, #130093546).

A monolayer of HEK293 expressing Covid-19 spike protein was prepared in a z-Movi® chip (LUMICKS®) coated with Poly-L-Lysine (Poly-L-Lysine (Sigma-Aldrich, cat. #P4707). A cell concentration of 60 million cells/mL resulted in a confluent monolayer. The monolayer cells were incubated for 2 hours to allow attachment to the z-Movi® chip surface. The monolayer stability was tested and was found to be stable when the cells were subjected repeatedly to a maximal force of 1000 pN (6 times in total) without significant loss of confluency. To allow differentiation between the B cells and the monolayer cells, the B cells were stained with Far Red CellTrace dye (Thermo Fisher, #C34564).

Then, the B cells were inserted in the z-Movi® chip and incubated for 15 minutes on the monolayer of HEK293 cells expressing Covid-19 spike protein. After incubation, the z-Movi® Cell Analyzer (LUMICKS®) applied a force ramp from 0 pN to 1000 pN in 2.5 minutes.

In a separate experiment, the procedure was repeated and included a blocking step of the B cells prior to insertion in the z-Movi® chip. B cells were incubated with soluble Covid-19 spike protein (Cat #293-cov2-s, obtained from Invivogen, SARS-COV-2 spike (D614)) for 5 minutes and then exposed to the monolayer cells. The spike protein was dissolved in PBS according to the manufacturer's instructions. B cells that remained bound to the HEK293 cells after the force was applied, were optionally stained with a labelled spike protein (Spike S1, AVI-HIS-TAG, iFluor-488-labeled (sars-Cov-2) recombinant (BPS Bioscience, #100936).

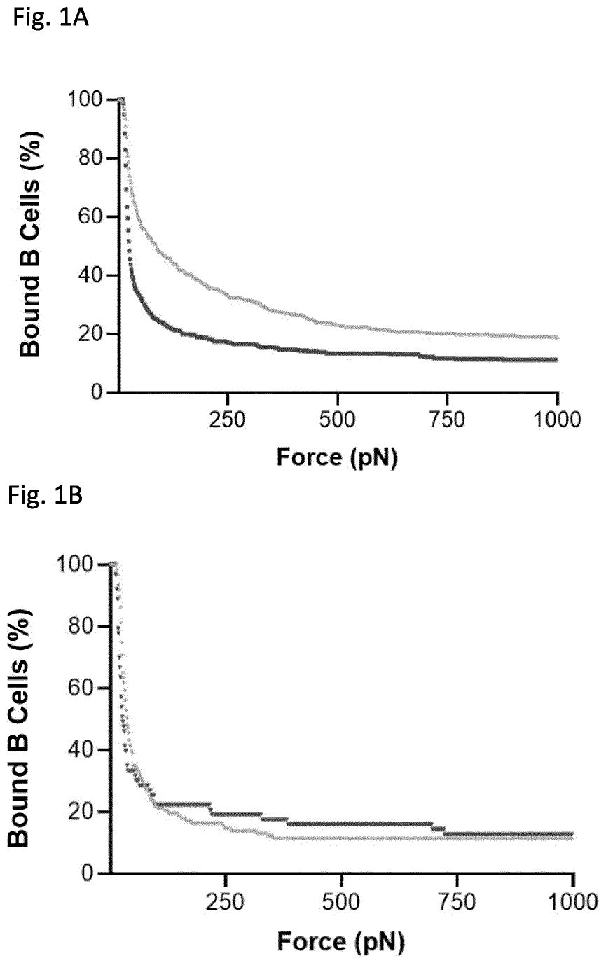

Experiments were conducted with 3 donors and similar results were obtained with the three different donors. Representative results are shown herein. FIG. 1 shows a plot depicting B cell detachment from HEK cells, from a single blood donor, with different experimental conditions. Further representative results obtained from measurements with B cells and calculations from a single donor are shown below in the tables.

TABLE 1
Percentage Memory or Non-Memory B cells remaining at the monolayer
of HEK293 cells expressing spike protein expressing relative to force
exerted under non-blocking conditions and blocking conditions with
soluble spike protein prior to contacting with the monolayer.

| | Memory | Non-Memory | Memory | Non-memory |
|---|---|---|---|---|
| | B cell | B cell | B cell | B cell |
| Force | (M) | (NM) | (M) | (NM) |
| (pN) | Non-blocking (NB) | | Blocking with spike protein (B) | |
| 0 | 100 | 100 | 100 | 100 |
| 125 | 48 | 21.4 | 20.3 | 22.2 |
| 250 | 37.8 | 17 | 14.6 | 19 |
| 375 | 34.4 | 14.7 | 11.4 | 17.5 |
| 500 | 32.1 | 13.3 | 11.4 | 15.9 |
| 625 | 31.5 | 13 | 11.4 | 15.9 |
| 750 | 30.7 | 11.6 | 11.4 | 12.7 |
| 875 | 30.4 | 11.4 | 11.4 | 12.7 |
| 1000 | 30.4 | 11.1 | 11.4 | 12.7 |

TABLE 2
Differences (Delta) calculated between percentages of
Memory B cells (M) and/or Non-Memory B cells (NM) bound,
with (B) and without blocking (NB) conditions.

| | Memory B cell | Non-Memory B cell | | |
|---|---|---|---|---|
| Force | (M) | (NM) | Delta M − NM | |
| (pN) | Delta NB − B | Delta NB − B | NB | B |
| 125 | 27.7 | −0.8 | 26.6 | −1.9 |
| 250 | 23.2 | −2 | 20.8 | −4.4 |
| 375 | 23 | −2.8 | 19.7 | −6.1 |
| 500 | 20.7 | −2.6 | 18.8 | −4.5 |
| 625 | 20.1 | −2.9 | 18.5 | −4.5 |
| 750 | 19.3 | −1.1 | 19.1 | −1.3 |
| 875 | 19 | −1.3 | 19 | −1.3 |
| 1000 | 19 | −1.6 | 19.3 | −1.3 |

TABLE 3
Ratios calculated between percentages of Memory
B cells (M) and/or Non-Memory B cells (NM) bound,
with (B) and without blocking (NB) conditions.

| | Memory B cell | Non-Memory B cell | |
|---|---|---|---|
| Force | (M) | (NM) | Ratio M:NM |

| (pN) | Ratio NB:B | Ratio NB:B | NB | B |
|---|---|---|---|---|
| 125 | 2.36 | 0.96 | 2.24 | 0.91 |
| 250 | 2.59 | 0.89 | 2.22 | 0.77 |
| 375 | 3.02 | 0.84 | 2.34 | 0.65 |
| 500 | 2.82 | 0.84 | 2.41 | 0.72 |
| 625 | 2.76 | 0.82 | 2.42 | 0.72 |
| 750 | 2.69 | 0.91 | 2.65 | 0.90 |
| 875 | 2.67 | 0.90 | 2.67 | 0.90 |
| 1000 | 2.67 | 0.87 | 2.74 | 0.90 |

The large delta for memory B cells comparing NB and B conditions (M, Delta NB-B) and the large delta for non-blocking condition comparing memory and non-memory B cells (Delta M-M, NB) indicates that a large portion of the memory B cell population have a specific interaction with the target cells expressing spike protein. Likewise, the same applies when looking at the calculated ratios. This was confirmed when staining remaining memory B cells after applying the force ramp with labelled spike protein.

FIG. 2 shows a result of a memory B cell bound to the monolayer after a force ramp was applied and stained with labelled spike protein. In contrast, when comparing blocking and non-blocking conditions for non-memory B cells, the delta was small (NM, Delta NB-B), and likewise, under blocking conditions the difference between memory B cells and non-memory B cells was small as well (Delta M-NM, B).

The results indicate that donor blood provided by Sanquin, obtained from three different donors, comprises a substantial portion of memory B cells having a high cellular avidity specific for covid-19 spike protein. Results indicate that cellular avidity can be utilized for manipulating memory B cells carrying a BCR of interest, i.e. specifically interacting with a target antigen presented on a cell surface.

This indicates that cellular avidity can be utilized to enrich and/or select for B cells with BCR receptors having desirable properties and/or identify sequences of interest of BCR receptors therefrom. These results indicate cellular avidity can be a valuable tool for the antibody discovery field.

This also indicates that cellular avidity can be utilized determining immune responses and specificity thereof. These results indicate cellular avidity can also be a valuable tool for immunology, in particular related to B cell immunology. Furthermore, these results indicate that cellular avidity methods can be a highly valuable tool in diagnostics, and for development of vaccine technology as well as in means and methods for assessing immune suppression or function.

### Example 2

With regard to separation or enrichment of different B cell clone populations, the concept of separating the cells utilizing cellular avidity separation methods is herein assessed. For example, a heterogeneous B cell population comprising 5 different cell clones, representing B cells obtained from a subject, having different cellular avidities with a target cell is provided. The comprised B cell clones A, B, C, D and E, each having a cellular avidity such that under the experimental conditions 95%, 75%, 50%, 25% and 10%, of the respective B cell clone population remains bound. When such B cells from a subject, consisting of 1000 cells, wherein each cell clone is present in specified amounts (w, x in table below), would be subjected to a cellular avidity separation and analysis in accordance with the invention, what would be the result?

Below in Table 4 the result is shown as obtained when fractions of detached cells (y) and cells that remained attached (z) would be collected and identified (e.g. with sequencing). In the table below, the % of cells at the start is listed, which can be determined e.g. by (sequence) analyzing a representative sample therefrom at the start, or calculating it from the total number of cells that were contacted with the target cells e.g. by adding detached and attached cell numbers.

TABLE 4
Cellular avidity data.

| | | % Cells | # Cells | Cells | Cells | % in | % in |
|---|---|---|---|---|---|---|---|
| | Cellular | Start | start | detached | attached | detached | attached |
| | avidity | w | x | y | z | q | r |
| A | 95% | 10% | 100 | 5 | 95 | 1% | 21% |
| B | 75% | 30% | 300 | 75 | 225 | 14% | 49% |
| C | 50% | 10% | 100 | 50 | 50 | 9% | 11% |
| D | 35% | 15% | 150 | 97 | 53 | 18% | 11% |
| E | 10% | 35% | 350 | 315 | 35 | 58% | 8% |
| Total | | | 1000 | 542 | 458 | | |

From this table, it is clear the number of cells attached or detached, or percentages thereof (see columns y, z, q and r) are not, by themselves, indicative for cellular avidity, which in this case, is known a priori. Hence, some calculations are needed from the results obtained that relate to cellular avidity. We can calculate for example the ratio of cells attached by cells at the start (z/x) to provide for a cellular avidity score. This number may also be expressed as percentages. This number can also be calculate based on the detached number of cells (1-y/x). This may also be referred to as an inferred absolute cellular avidity, as opposed to relative cellular avidity (see below).

We can also calculate a relative cellular avidity, if we for example do not know the number of cells that interacted with the cell target layer. This number can be calculated by calculating the ratio of % of cells detached divided by the % of cells at the start (r/w). This number reflects the fold enrichment obtained in the fraction. One can also calculate the ratio of cells attached by the cells detached (r/q or z/y). Besides cellular avidity, this number also indicates the fold enrichment of the attached fraction relative to the detached fraction. The results of these calculations are listed below. Whereas in the above table no ranking could be made, the calculated (relative/absolute) cellular avidity scores rank as they should.

TABLE 5
Calculations of cellular avidity.

| | | Absolute | Relative cellular | Relative cellular |
|---|---|---|---|---|
| | Cellular | Cellular avidity | avidity | avidity |
| | avidity | z/x | z/y | r/w |
| A | 95% | 0.95 | 19.00 | 2.08 |
| B | 75% | 0.75 | 3.00 | 1.64 |
| C | 50% | 0.50 | 1.00 | 1.09 |
| D | 35% | 0.35 | 0.54 | 0.77 |
| E | 10% | 0.10 | 0.11 | 0.22 |

The above shows that when the composition of at least two fractions is either known or determined, cellular avidity can be determined which allows for ranking identified B cell clones, and their corresponding target cell. Hence, the above shows that when B cell clones are identified, e.g. by sequencing the heavy and/or light chain sequences, and quantified, from B cells obtained from a subject, one can easily determine cellular avidity and rank B receptors, or candidate antibody sequences, accordingly.

1. A method for enrichment of B cells expressing an antigen-specific BCR comprising the steps of:
a) providing B cells obtained from a subject,
b) providing target cells expressing an antigen, attached to a surface,
c) contacting the B cells with the target cells attached to a surface, to allow the B cells to interact with the target cells,
d) exerting a force on the B cells, wherein the force is in a direction away from the target cells, and
e) removing unbound B cells and/or collecting the B cells that remained attached to the target cells.

2. The method in accordance with claim 1, wherein the provided B cells are obtained from a subject exposed to the antigen.

3. The method in accordance with claim 1, wherein the B cells that remained attached to the target cells after step d) are collected and further enriched, optionally using FACS or MACS.

4. A method for identifying B cells expressing an antigen-specific BCR comprising the steps of:
a) providing B cells obtained from a subject exposed to an antigen,
b) providing target cells expressing the antigen attached to a surface,
c) contacting the B cells with the target cells attached to a surface to allow the B cells to interact with the target cells,
d) exerting a force on the B cells, wherein the force is in a direction away from the target cells,
e) providing a labelled antigen,
f) staining the B cells that remained attached to the target cells with a labelled antigen;
therewith identifying remaining B cells expressing antigen-specific BCRs, and
g) optionally, collecting the identified B cells expressing antigen-specific BCRs.

5. The method in accordance with claim 1, comprising a step of collecting B cells, and the collected B cells are further cultured and/or immortalized.

6. The method in accordance with claim 1, wherein the sequences of the variable regions of the heavy chain and/or light chain expressed by the collected B cells are determined.

7. A method for identifying B cells expressing an antigen-specific BCR comprising the steps of:
a) providing B cells obtained from a subject exposed to an antigens,
b) optionally, determining of a sample of the B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells and/or that detached from the target cells,
g) determining the sequences of the variable region of the heavy chain and/or the light chain expressed by the collected B cells,
h) using the determined sequences to identify B cell clones and determine the number of B cells representing a B cell clone of the B cells that remained attached to the target cells and/or detached from the target cells, and, optionally, the number of B cells representing a B cell clone in the sample, and
i) identifying a B cell expressing an antigen-specific BCR based on the determined numbers by identifying B cell clones that are enriched in the fraction representing B cells attached to the target cells.

8. A method for determining a cellular avidity score of B cell clones with target cells expressing an antigen comprising the steps of:
a) providing B cells obtained from a subject exposed to an antigen,
b) optionally, determining of a sample of the provided B cells the sequences of the variable regions of the heavy chain and/or light chain expressed by the B cells,
c) providing target cells expressing an antigen attached to a surface,
d) contacting the B cells with the target cells attached to a surface to allow the B cells to interact with the target cells,
e) exerting a force on the B cells, wherein the force is in a direction away from the target cells,
f) collecting the B cells that remained attached to the target cells, and/or, collecting B cells that detached from the target cells,
g) determining the sequences of the variable region of the heavy chain and/or the light chain expressed by the collected B cells,
h) using the determined sequences to identify B cell clones and determine the number of B cells representing a B cell clone of the B cells that remained attached and/or detached from the target cells, and optionally, the number of B cells representing a B cell clone in the sample,
i) assign a cellular avidity score to identified B cell clones based on the determined numbers.

9. Method in accordance with claim 7, wherein the B cells that remained attached are stained with one or more labelled antigens.

10. Method in accordance with claim 1, wherein the B cell is a memory B cell, preferably CD27+.

11. Method in accordance with claim 1, wherein the B cells are obtained from blood, preferably from a human.

12. Method in accordance with claim 1, wherein the force that is applied is in the range of 1 pN-10 nN.

13. Method in accordance with claim 1, wherein the force is an acoustic force, a shear flow force or a centrifugal force.

14. The method in accordance with claim 1, wherein in the step of applying a force on the B cells away from the target cells, instead a differential force is applied which does not require attachment of the target cells to a surface, and, wherein said target cells are optionally attached to a surface.

15. The method in accordance with claim 1, wherein the antigen is selected from the group of cancer antigens, pathogen antigens such as viral antigens and bacterial antigens, and allergens.
